(19) United States
(12) Patent Application Publication
(10) Pub. No.: US 2026/0091106 A1
(43) Pub. Date: Apr. 2, 2026

(54) COMPOSITIONS AND METHODS FOR MAKING AND USING STABILIZED ENVELOPED VIRUSES BY SPRAY-DRYING AND LYOPHILIZATION

(71) Applicant: THE REGENTS OF THE UNIVERSITY OF COLORADO, A BODY CORPORATE, Denver, CO (US)

(72) Inventors: Holly COLEMAN, Denver, CO (US); Theodore W. RANDOLPH, Denver, CO (US); Daniel K. SCHWARTZ, Denver, CO (US); Robert GARCEA, Denver, CO (US)

(21) Appl. No.: 19/348,562

(22) Filed: Oct. 2, 2025

Related U.S. Application Data
(63) Continuation of application No. PCT/US2024/022887, filed on Apr. 3, 2024.
(60) Provisional application No. 63/456,720, filed on Apr. 3, 2023.

(51) Int. Cl. A61K 39/21; A61K 9/16; A61K 9/19; A61K 39/205; A61K 47/18; A61K 47/22; C12N 7/00

(52) U.S. Cl. CPC A61K 39/21; A61K 9/1623; A61K 9/1652; A61K 9/19; A61K 39/205; A61K 47/183; A61K 47/22; C12N 7/00; C12N 2710/14034; C12N 2740/15034; C12N 2760/20034

(57) ABSTRACT

Embodiments of the present invention provide for novel compositions and methods for making and using thermostable enveloped viruses. Certain embodiments relate to formulating enveloped viruses alone in a aqueous formulation, or in combination with spray-drying, lyophilization or other drying techniques to reduce degradation and/or preserve activity or titer of the enveloped virus reducing titer loss, prolonging storage stability, delivery, and use. In some embodiments, formulations disclosed herein create a stabilized envelope virus formulation for protection from high temperature during drying processes for an enhanced and rapid drying process for storage, transport and later use of the stabilized envelope viruses.

### PRIORITY

This Application is a continuation application that claims priority to International Application No. PCT/US2024/022887, filed Apr. 3, 2024, which claims priority to U.S. Provisional Application No. 63/456,720 filed Apr. 3, 2023. These applications are incorporated herein by reference in their entireties for all purposes.

### FIELD

Embodiments of the present invention provide for novel compositions and methods for making and using stable enveloped viruses. Certain embodiments relate to formulating enveloped viruses alone or in combination with spray-drying, lyophilization, or other drying techniques to reduce degradation and/or preserve activity or titer of the enveloped virus prolonging storage, delivery, and use in aqueous or essentially dry formulations.

### BACKGROUND

Viruses can be classified into two broad categories based on their structure as enveloped or non-enveloped viruses. Non-enveloped viruses are made up of a protein capsid surrounding RNA or DNA. Enveloped viruses have the same components as non-enveloped viruses but are surrounded by a lipid bilayer (known as the envelope) which is derived from a host-cell membrane. Surface proteins involved in cell infection typically protrude through the envelope of these viruses. In general, enveloped viruses are considered to be less stable and more sensitive to thermal stress and desiccation processes than non-enveloped viruses, in part because the lipid bilayer of an enveloped virus is less robust than the protein capsid of a non-enveloped virus.

Whole live and attenuated viruses have been used in vaccines and other therapies for many years. Recently, interest has aroused regarding using live viruses for delivery of genetic material in immunotherapies designed to treat cancer and other diseases. However, the instability of enveloped viruses typically imposes stringent cold chain requirements for most viral products during manufacturing, storage, and distribution prior to administration to patients, making use of these enveloped viruses unpredictable and in certain cases unreliable due to their susceptibility to degradation. This issue frequently creates the need for cold storage and often thawing and immediate use of frozen compositions to avoid degradation of the product prior to administration. Therefore, a need exists to develop compositions and/or methods for improving stability and retaining viability of these enveloped viruses.

### SUMMARY

Embodiments of the present invention provide for novel compositions and methods for making and using stable enveloped viruses. Certain embodiments relate to formulating enveloped viruses alone or in combination with spray-drying, lyophilization or other drying techniques to reduce degradation and/or preserve activity or titer of the enveloped virus prolonging storage, facilitating manufacture, delivery, and use. In certain embodiments, formulations containing enveloped viruses prepared in compositions and by methods disclosed herein can be used to produce therapeutics for treating, ameliorating and/or preventing a health condition.

In certain embodiments, an enveloped virus-containing composition or formulation disclosed herein can include, but is not limited to, one or more enveloped viruses; one or more non-reducing saccharide agents; one or more polysaccharide agents; and at least one amino acid. In certain embodiments, the composition can further include a buffering agent. In some embodiments, the compositions or formulation can further include one or more volatile salts. In some embodiments, the enveloped virus composition is essentially dried.

In certain embodiments, an enveloped virus-containing composition or formulation disclosed herein can include, but is not limited to, one or more polysaccharides including, but are not limited to, one or more of hydroxyethyl starch, hydroxypropyl starch, amylose, amylopectin, cellulose, methylcellulose, carboxymethylcellulose, dextrin, cyclodextrin or a combination thereof. In other embodiments, the enveloped virus-containing composition can include one or more non-reducing saccharides including, but not limited to, trehalose, sucrose, lactose, raffinose, gentiarose, melezitose, stachyose, and verbascose or a combination thereof. In some embodiments, the enveloped virus-containing composition or at least one amino acid of use in formulations or compositions disclosed herein can include, but is not limited to, histidine, methionine, arginine, proline, glycine, isoleucine, leucine, or the like, or other amino acid.

In certain embodiments, an enveloped virus-containing composition or formulation disclosed herein can include, but is not limited to, one or more volatile salts including one or more of ammonium acetate, ammonium formate, ammonium carbonate, ammonium bicarbonate, triethylammonium acetate, triethylammonium formate, triethylammonium carbonate, trimethylamine acetate trimethylamine formate, trimethylamine carbonate, pyridinal acetate, and pyridinal formate, or combinations thereof. In other embodiments, an enveloped virus-containing composition or formulation disclosed herein does not include a volatile salt. In certain embodiments, an enveloped virus-containing composition or formulation disclosed herein does not include ammonium acetate.

In certain embodiments, an enveloped virus-containing composition or formulation disclosed herein can be exposed to elevated temperatures wherein the enveloped virus(es) remains stable with reduced degradation and reduced loss of titer. In some embodiments, the enveloped virus-containing composition or formulation disclosed herein can be exposed to elevated temperatures during the process by which the enveloped virus-containing composition or formulation is dried. In accordance with these embodiments, elevated temperatures of exposure for these compositions or formulations during the drying process can include, but are not limited by temperatures ranging from about 80° C. to about 90° C.; 70° C. to about 90° C.; about 60° C. to about 90° C.; about 55° C. to about 90° C.; about 50° C. to about 90° C.; about 45° C. to about 90° C.; about 35° C. to about 90° C.; or about 30° C. to about 90° C. In some embodiments, the enveloped virus-containing composition or formulation disclosed herein can be exposed to elevated temperatures after the enveloped virus-containing composition or formulation is dried such as by lyophilization or spray-drying or similar drying processes wherein the enveloped virus(es) remains stable with reduced degradation. In accordance with these embodiments, elevated temperatures of exposure for these compositions or formulations after lyophilization or spray-drying or other drying procedure contemplated herein can include but are not limited to, temperatures ranging from about 4° C. to about 100° C., 4° C. to about 80° C.; about 4° C. to about 60° C.; about 4° C. to about 55° C.; about 4° C. to about 50° C.; about 4° C. to about 45° C.; about 4° C. to about 40° C.; about 4° C. to about 35° C.; about 4° C. to about 30° C. or about 4° C. to about 25° C. for at least one hour to several days, several weeks or several months depending on the temperature of exposure and conditions for storage.

In certain embodiments, an enveloped virus-containing composition or formulation disclosed herein can include one or more enveloped virus(es). In accordance with these embodiments, the one or more enveloped virus(es) can include one or more DNA viruses, RNA viruses, or a combination thereof. In certain embodiments, the one or more DNA virus(es) can include, but are not limited to, baculoviruses, poxviridae, fowlpox virus, herpes viruses, poxviruses, hepadnaviruses, asfarviruses, other DNA viruses of use in therapeutic applications where formulations and methods disclosed herein can stabilize the enveloped viruses and a combination thereof. In certain embodiments, the one or more RNA virus(es) can include, but are not limited to, lentiviruses, flaviviruses (e.g., dengue, yellow fever, Zika virus), lyssaviruses, ephemeroviruses, and Vesiculovirus, alphaviruses, togaviruses, coronaviruses, hepatitis D, orthomyxoviruses, paramyxoviruses, avian paramyxoviruses rhabdovirus, bunyaviruses, influenza, hepatitis C, measles, Vesicular stomatitis, mumps, and Ebola virus, other filoviruses and a combination thereof.

In certain embodiments, an enveloped virus-containing composition or formulation disclosed herein can include one or more enveloped viruses including, but not limited to, one or more retroviruses. In accordance with these embodiments, the one or more retroviruses can include one or more RNA tumor viruses. In other embodiments, the one or more retroviruses can include, but is not limited to, human immunodeficiency virus (HIV) or other lentiviruses, human T-lymphotropic virus type 1 (HTLV-1), human T-lymphotropic virus type 2 (HTLV-II) or other oncoretrovirus, or other retrovirus known, or unknown in the art.

In certain embodiments, an enveloped virus-containing composition or formulation disclosed herein can include one or more enveloped viruses including, but not limited to, one or more of influenza A virus, influenza B virus, influenza C virus, influenza D virus, the respiratory syncytial virus (RSV), corona virus, hepatitis B, hepatitis C, Ebola virus, lentiviruses In certain embodiments, compositions and formulations disclosed herein can be used to stabilize vaccines employing attenuated or killed enveloped viruses or other enveloped viruses use for therapeutic purposes. In certain embodiments, compositions and formulations disclosed herein can be used to stabilize vaccines employing attenuated, killed, or inactivated enveloped viruses. In some embodiments, killed rabies viruses can be stabilized by compositions and methods disclosed herein, leading to vaccine formulations that require minimal or no refrigeration for transport, storage, and delivery to a subject. In certain embodiments, compositions and formulations disclosed herein can be used to generate CAR-T cells produced by enveloped viruses contemplated herein providing improved and more efficient outcomes by stabilizing the viruses used to produce the CAR-T cells for therapeutic purposes. In some embodiments, CAR-T cells can be produced using lentiviruses stabilized by compositions and methods disclosed herein.

In certain embodiments, a pharmaceutical composition including, but not limited to, a composition or formulation disclosed herein is contemplated where the composition or formulation further includes at least one pharmaceutically acceptable excipient. In accordance with these embodiments, a pharmaceutical composition or formulation disclosed herein can include re-constituting or re-hydrating an essentially dried enveloped virus-containing composition or formulation disclosed herein. In certain embodiments, a reconstituted or rehydrated formulation disclosed herein can be used as a prophylactic, for ameliorating a condition or for treating a condition disclosed herein. In some embodiments, a reconstituted or rehydrated formulation disclosed herein can be used as a vaccine to reduce onset or prevent a health condition.

Other embodiments, methods are disclosed for creating and using stabilized enveloped virus compositions and formulations disclosed herein. In one embodiment, the method includes, but is not limited to, combining one or more enveloped viruses with a formulation; the formulation including, but not limited to, one or more non-reducing disaccharide agent(s); one or more starches; one or more amino acid; and generating a liquid enveloped virus formulation. In certain embodiments, the enveloped virus composition or formulation can further include one or more volatile salts. In certain embodiments, the method can further include freezing the liquid enveloped virus formulation; and lyophilizing the liquid enveloped virus formulation creating an essentially dry powder of an enveloped virus formulation. In other embodiments, the method can further include spray-drying the liquid enveloped virus formulation; and creating an essentially dry powder of an enveloped virus formulation. In some embodiments, the essentially dry powder of enveloped virus formulation can be stored at elevated temperatures for transport or later use. In yet other embodiments, the method can further include re-constituting or re-hydrating the essentially dried formulation or composition.

Other embodiments, kits for preparing, storing, or delivering formulations and compositions are disclosed herein. In certain embodiments, kits can include at least one container. In other embodiments, kits can include formulations and compositions disclosed herein for making, storing, or using one or more enveloped virus and optionally, a device for delivering the one or more enveloped virus formulation to a subject to prevent, ameliorate, or treat a condition.

### BRIEF DESCRIPTION OF THE FIGURES

The following drawings form part of the instant specification and are included to further demonstrate certain aspects of particular embodiments herein. The embodiments may be better understood by reference to one or more of these drawings in combination with the detailed description presented herein. The accompanying drawings are incorporated into and form a non-limiting part of the specification to illustrate several examples of the present disclosure.

### BRIEF DESCRIPTION OF THE FIGURES

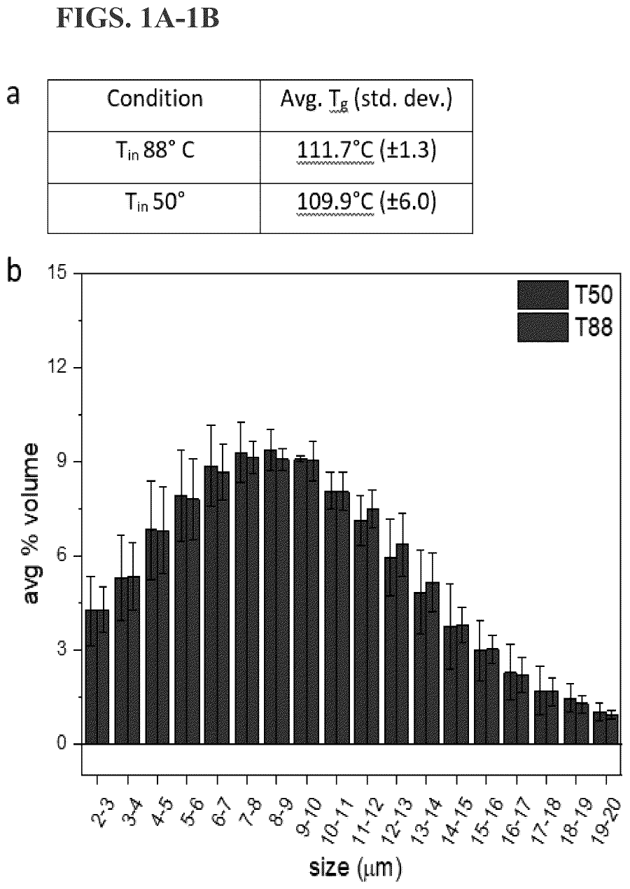

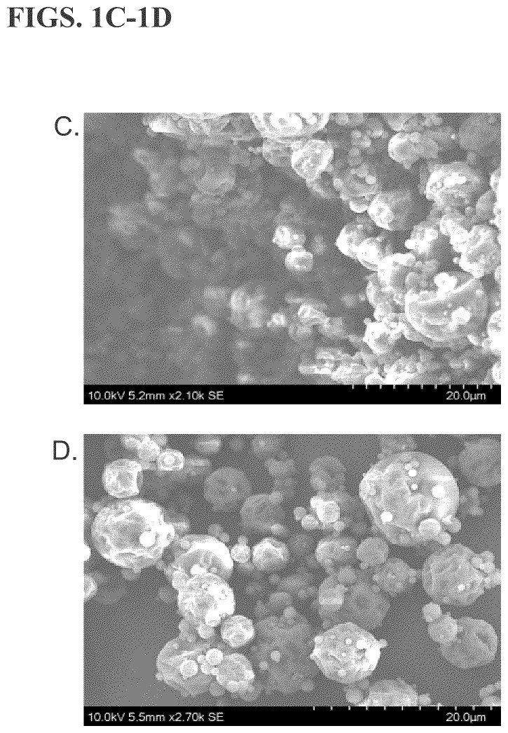

FIG. 1A is a table illustrating an exemplary example of glass transition temperature (Tg) of a formulation of spray dried experimental enveloped virus under different drying conditions in accordance with certain embodiments disclosed herein.

FIG. 1B illustrates particle size distribution of spray dried experimental formulation of enveloped viruses in formulations disclosed herein under different drying conditions showing nearly indistinguishable differences in accordance with certain embodiments disclosed herein.

FIGS. 1C-1D illustrate scanning electron microscopy (SEM) of spray dried formulation of experimental enveloped viruses spray-dried at various inlet temperatures and the essentially-dried particles compared in accordance with certain embodiments disclosed herein.

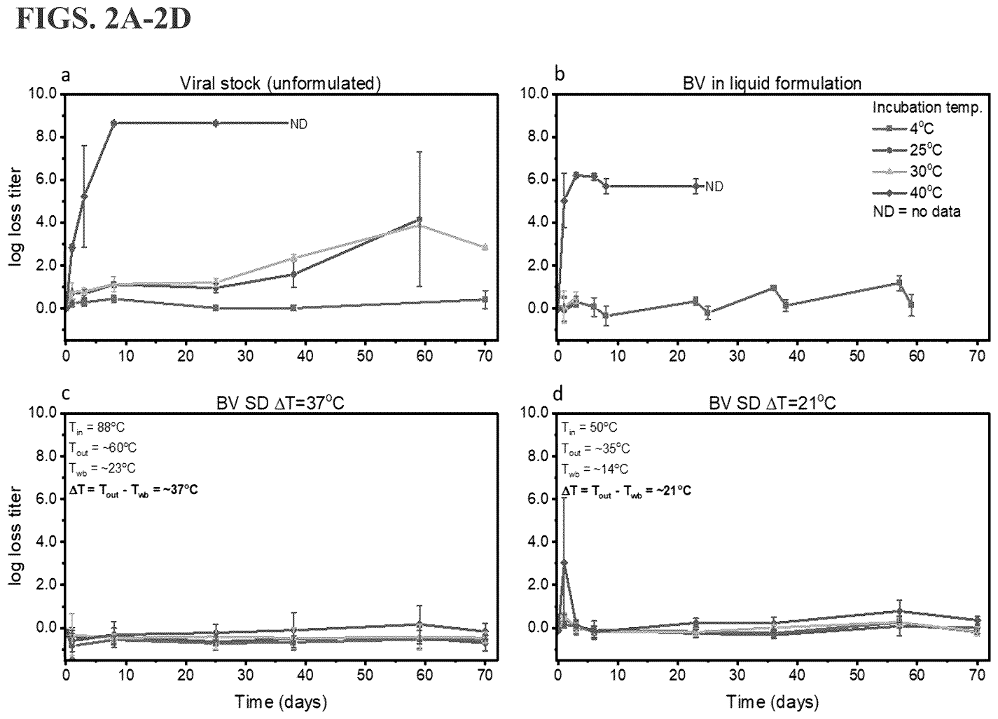

FIGS. 2A-2D represents exemplary graphs illustrating titer loss of experimental enveloped viruses under various conditions of unformulated (FIG. 2A), formulated (FIG. 2B) aqueous samples, or essentially dried (FIGS. 2C-2D) following a predetermined incubation period under various temperatures in accordance with certain embodiments disclosed herein.

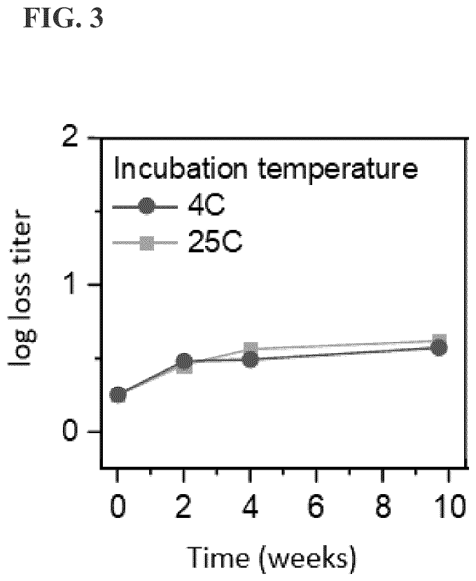

FIG. 3 represents an exemplary graph illustrating titer loss of experimental enveloped viruses over a pre-determined period at different temperatures in accordance with certain embodiments disclosed herein.

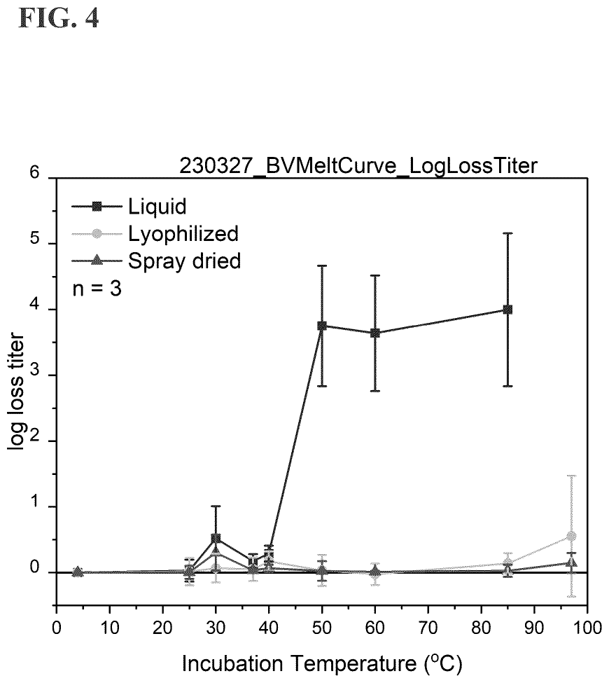

FIG. 4 represents an exemplary graph comparing log loss of titer of formulated, spray-dried, and lyophilized exemplary enveloped virus incubated for one hour at various temperatures. An exemplary enveloped virus in liquid formulation (purple squares), spray-dried (green triangles) and lyophilized (light blue circles) were studied in accordance with certain embodiments disclosed herein.

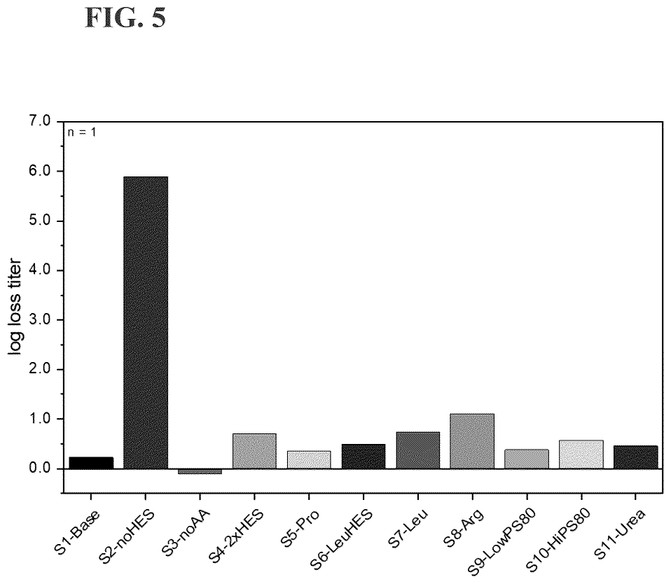

FIG. 5 represents an exemplary bar graph comparing viral titer loss of experimental enveloped viruses under various formulation conditions and incubated under pre-determined conditions in accordance with certain embodiments disclosed herein.

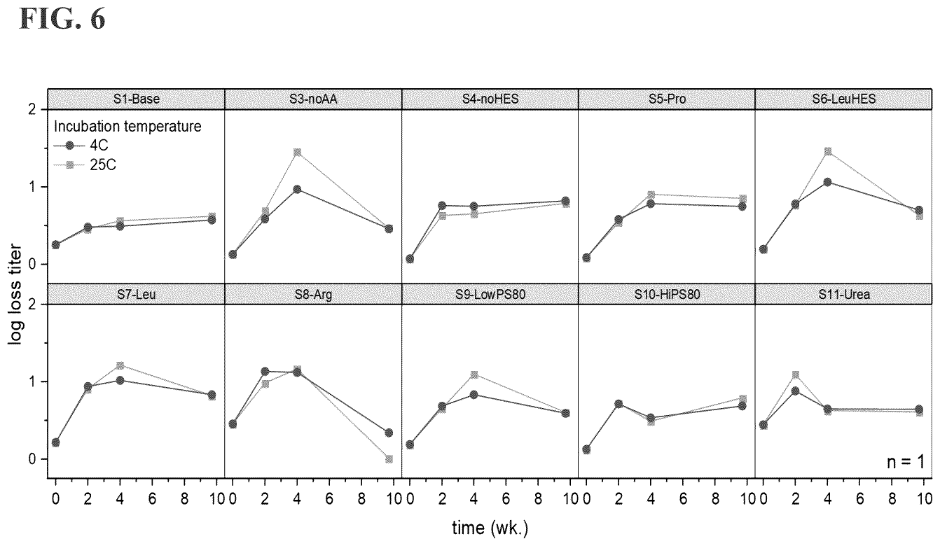

FIG. 6 represents exemplary graphs comparing viral titer loss over time of experimental enveloped viruses under various formulation conditions and incubated under pre-determined conditions at the illustrated temperatures in accordance with certain embodiments disclosed herein.

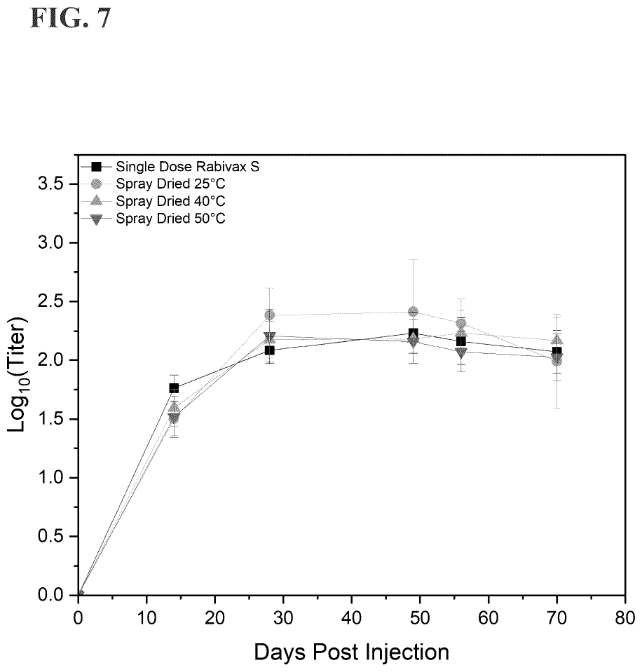

FIG. 7 represents an exemplary graph comparing viral titer loss over time of experimental enveloped viruses under various formulation conditions and incubated under pre-determined conditions at the illustrated temperatures in accordance with certain embodiments disclosed herein.

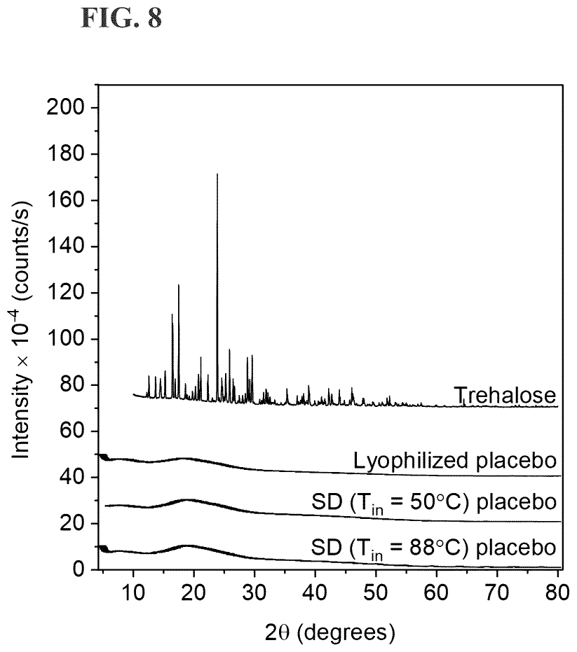

FIG. 8 represent exemplary graphs represents exemplary graphs comparing XRD spectra of spray-dried and lyophilized placebo samples and unprocessed trehalose in accordance with certain embodiments disclosed herein.

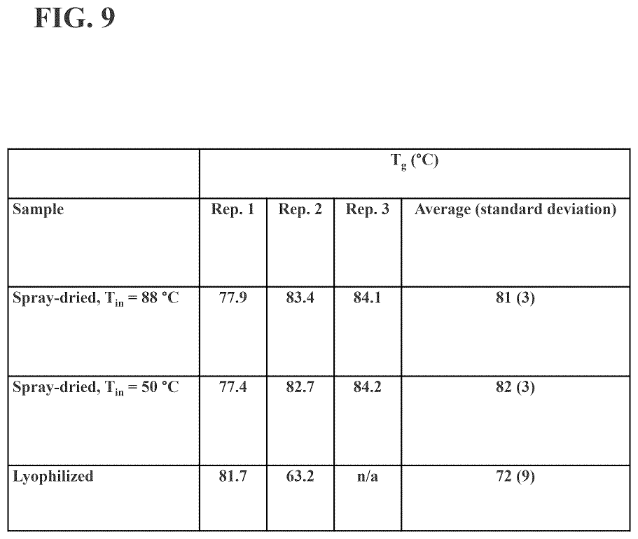

FIG. 9 represents an exemplary table where glass transition temperatures Tg were measured for placebo spray-dried powders and lyophilized cake in accordance with certain embodiments disclosed herein.

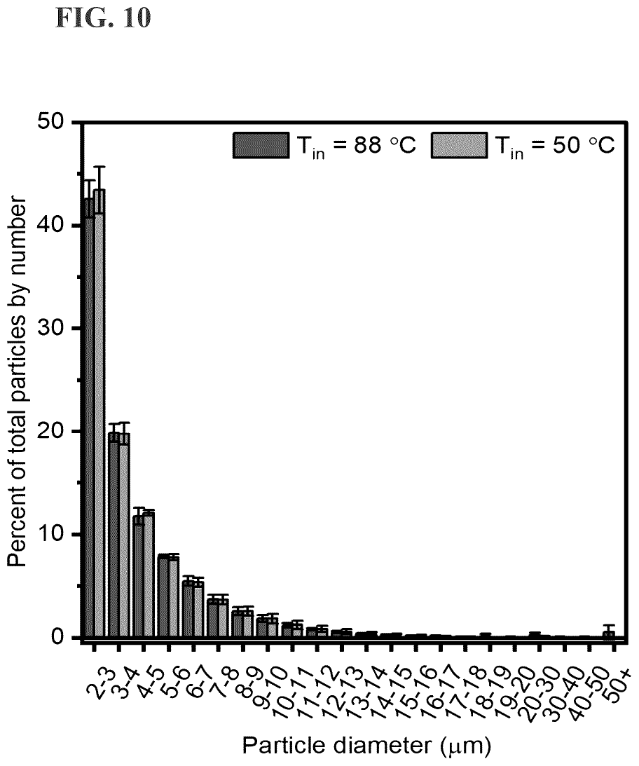

FIG. 10 represents an exemplary bar graph comparing particle size distribution of spray-dried particles measured by FIM. Particle size distributions are presented for particles formed by spray-drying using heated air with a temperature of 88° C. (blue bars, left bars) or 50° C. (orange bars, right bars for each distribution analyzed) in accordance with certain embodiments disclosed herein.

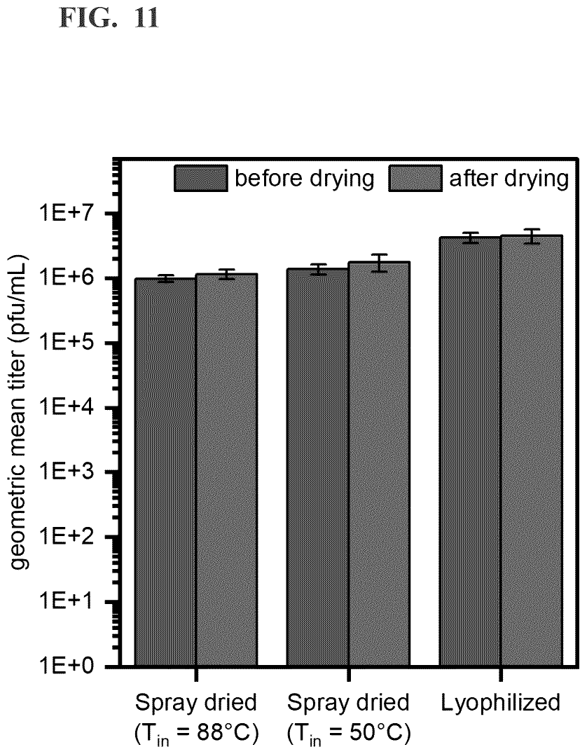

FIG. 11 represents an exemplary bar graph comparing viral titer of enveloped virus in liquid formulations prior to drying (blue bars, left bars) and in reconstituted lyophilized or spray-dried formulations (green bars, right bars). Titers before (blue bars, left) and after (green bars, right of each set) in accordance with certain embodiments disclosed herein.

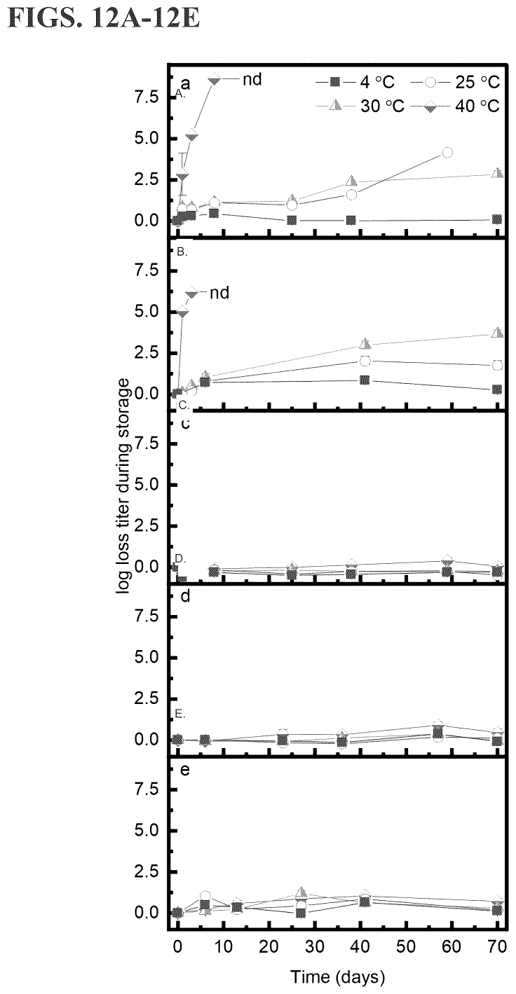

FIGS. 12A-12E represent exemplary graphs comparing log loss titers for enveloped virus during storage in liquid, spray-dried powders, and lyophilized cakes. Enveloped virus was incubated at 4° C. (blue squares), 25° C. (green circles), 30° C. (orange triangles) or 40° C. (red diamonds) as supplied (viral stock, A), after formulation (B), after spray-drying at a Tin of 88° C. (C) or 50° C. (D), and after lyophilization (E) in accordance with certain embodiments disclosed herein.

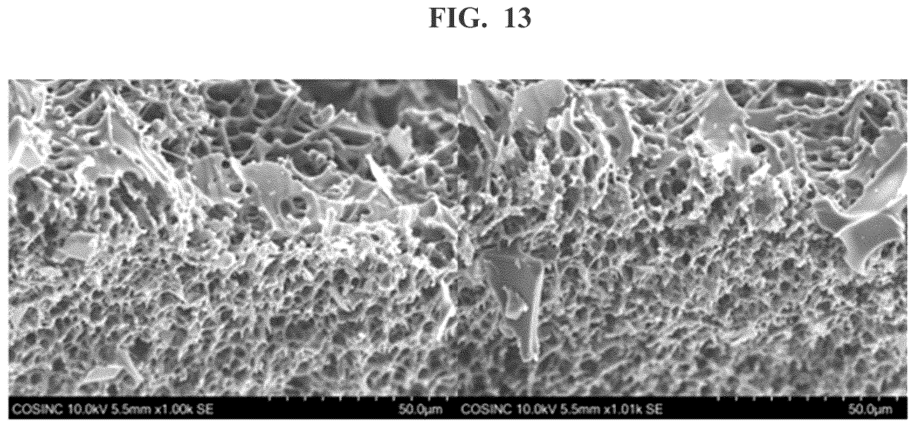

FIG. 13 represents an image of an SEM of lyophilized placebo formulation. Cross-sections of lyophilized placebo cakes were mounted on imaging stubs via double-sided carbon tape in accordance with certain embodiments disclosed herein.

### DEFINITIONS

In order to facilitate an understanding of the invention, the following definitions are provided.

As used herein, “a” or “an” may mean one or more than one of an item.

As used herein, “about” may mean up to and including plus or minus five percent, for example, about 100 may mean 95 and up to 105.

### DETAILED DESCRIPTIONS

In the following sections, various exemplary compositions and methods are described in order to detail various embodiments. It will be obvious to one skilled in the art that practicing the various embodiments does not require the employment of all or even some of the details outlined herein, but rather that concentrations, times and other details may be modified through routine experimentation. In some cases, well-known methods or components have not been included in this description.

Liquid formulations of viral products often require frozen storage between about −20° C. and −80° C. To reduce the burden of these cold chain transport and storage requirements, many viral products are lyophilized, for example, vaccines. However, even these lyophilized products often require refrigeration or frozen storage beyond a short-term exposure (e.g., minutes to about an hour) to ambient temperatures during transport, storage, or application. These disadvantages can contribute to product loss, viral titer loss, and an increased cost to manufacture and the need to maintain cold chain requirements impeding distribution of these products or impeding stockpiling of products such as vaccines or other therapies for emergency responses to a multitude of recipients at one time. In addition, lyophilization is often used to reduce cold chain requirements of viral products such as vaccines. However, lyophilization can still lead to stresses to viruses such as titer loss for example, osmotic stress due to cryo-concentration, intra-virus ice formation, adsorption to ice-water interfaces, aggregation, and pH shift in formulation. Further, lyophilization is an energy intensive process making it costly to perform. Other drying options include spray-drying for a more affordable scalable, continuous operation for production of essentially dry viral products with improved stability. However, large viral titer losses have been observed using spray-drying, and the mechanisms that lead to viral titer loss during spray-drying of viruses remain mostly unknown.

It is known that current gene therapies and immunotherapies often rely on infectious viruses as vectors. Gene therapy, which includes therapeutic manipulation of gene expression by correction, supplementation, or modification of specific genes, has benefited from the use of viral vectors for gene delivery due to their superiority compared to current non-viral vectors. Viral vectors, including adeno-associated viruses, adenoviruses, and lentiviruses, have demonstrated success in treating a myriad disorders including, but not limited to, cancer, cardiovascular diseases, immunodeficiencies, brain disorders, inflammatory disorders and more. Nearly two-thirds of the over 30 existing FDA-approved gene-based products use viral vectors to deliver genetic information into patient cells (e.g., HEMGENIX™ and ADSTRILADRIN™), or to engineer CAR T-cells ex vivo (e.g., Kymriah and YESCARTA™). Dozens of other promising gene therapy products that use viral vectors are currently in pre-clinical or clinical trials. Therefore, there is a need to supply and maintain viral vector stability for uses in these sectors. Compositions and methods disclosed herein provide improved formulations and processes to address viral vector instability issues.

In other embodiments, immunotherapy has been used to manipulate a subject's immune system to target cancer cells and treat infectious diseases. In accordance with these embodiments, T-VEC was the first FDA approved oncolytic virus for cancer therapy, using an engineered herpes simplex virus 1 to target malignant cells in patients with melanoma. No other oncolytic viruses have been approved by the FDA to date, but pre-clinical and clinical research suggests that many different types of viruses can be used for these therapies and others, either alone or in combination with other anti-cancer therapies.

In certain embodiments, live, attenuated viruses can be and have been used in vaccines such as measles, mumps, rubella, polio, and varicella. Live, attenuated viruses are potent vaccine antigens because they generate both cellular and humoral immune responses, do not require adjuvants, and typically require administration of only one or two doses for effective vaccination. A recent review of nearly 1,000 vaccines currently in development found that 23% of vaccine candidates are traditional inactivated or attenuated viral vaccines, with an additional 14% of vaccines using viral vectors. One disadvantage of viruses in vaccines and other therapeutics is their instability in liquid formulations, where viral components can be subjected to a variety of chemical and physical degradation pathways. Enveloped viruses are known to be less thermally stable than their non-enveloped counterparts. Refrigerated (2° C. to 8° C.) or frozen (−80° C. to −20° C.) storage is typically required to maintain the efficacy of viral products which increases cost and likelihood of degradation if not maintained with cold chain temperature ranges or used in a proscribed time. Cold chain maintenance for vaccines and virus-based therapeutics is a major challenge during their manufacturing, storage, and distribution, especially to low resource environments, which contributes to vaccine wastage and limits the equitable access to potentially life-saving viral therapies. Stringent temperature requirements also complicate manufacturing, storage, and delivery of viral gene and immunotherapies, contributing to the prohibitively high costs of these therapies.

In certain embodiments, solid dry formulations can be useful in stabilizing biologics, including viruses, against physical and chemical degradation pathways occurring in the liquid state. When biologics are embedded in glassy matrices and maintained at temperatures sufficiently below the glass transition temperature (Tg), molecular motions are inhibited such that these degradation pathways cannot occur or occur at dramatically reduced rates. Therefore, dry, glassy formulations can extend product shelf-life and ease or eliminate cold storage requirements if appropriate formulations for the targeted agent (e.g., enveloped viruses) are available. One method as contemplated herein, for embedding biologics into a glassy matrices is using a freeze-drying or lyophilization technique. In lyophilization, aqueous solutions or suspensions are first frozen, followed by removal of water by sublimation at low pressures and temperatures. Many live viral vaccines are lyophilized; however, these vaccines may suffer potency losses during lyophilization, and even after lyophilization vaccines frequently require storage at −20° C. or 2° C. to 8° C. Therefore, as an alternative, spray-drying can be used where a target agent is embedded within glassy matrices while avoiding potentially damaging freezing steps. In spray-drying, a bulk aqueous suspension is nebulized into a concurrently flowing, heated dry gas stream (typically heated air or nitrogen gas), removing water by evaporation. Spray-drying is a scalable, continuous operation that offers several practical advantages over lyophilization, including reduced energy and operating costs. However, large titer losses of viruses have sometimes been observed during spray-drying, and to date no spray-dried vaccines are on the market.

In certain embodiments, it was discovered that glassy, spray-dried formulations enhanced the thermal stability of certain virus-like particles, bacteriophages, and phage-like particles but formulations of use in these processes for the special features of enveloped viruses were unknown. Immunogenicity of the virus- and phage-like particles was retained through spray-drying in these formulations. As contemplated herein, incorporation of enveloped viruses into glassy matrices created by lyophilization, or spray-drying can enhance thermal stability of the enveloped viruses, similar to the effects of glassy-state immobilization of therapeutic proteins and lipid bilayers, without causing significant drying-induced loss of titer. Furthermore, compositions and methods disclosed herein using an infectious enveloped virus in the glassy matrix under these conditions were discovered to be not damaging to the encased infectious enveloped virus. As disclosed herein, spray-drying is an attractive alternative to lyophilization because it is a continuous operation that is less energy intensive and yields lower operating costs compared to lyophilization. Spray-drying also provides for the production of dosage forms that can be delivered by parenteral, nasal, and/or pulmonary routes. It has been observed that other formulations in the art led to high titer loss during spray-drying of viruses and viral vectors. In contrast using formulations disclosed herein, little to no loss of titer after spray-drying or lyophilization were observed using the presented formulations. As disclosed herein, formulated spray-dried or lyophilized enveloped virus are presented.

In certain embodiments, formulations disclosed herein include a surfactant. In other embodiments, formulations disclosed herein specifically omit a surfactant. In accordance with these embodiments, an omitted surfactant can include an omitted non-ionic surfactant. Compositions, formulations, and suspensions disclosed herein protect enveloped viruses from titer loss during either lyophilization or spray drying for prolonged storage and use at temperatures of or above room temperature.

In certain embodiments, enveloped viruses can be and have been demonstrated as attractive candidates for use as gene- and immunotherapeutic agents due to their efficacy at infecting host cells and delivering genetic information. They have also been used in vaccines as potent antigens to generate strong immune responses, often requiring fewer doses than other vaccine platforms as well as eliminating the need for adjuvants. Embodiments disclosed herein concern improving envelope virus stability in liquid formulations to enhance their shelf life for transport and storage. To date, enveloped viruses in liquid formulations typically lose infectivity within an hour at temperatures above 40° C., or after incubation at 25° C. for longer periods of time.

In some embodiments, provide for novel compositions and methods for making and using stable enveloped viruses. Certain embodiments relate to formulating enveloped viruses alone or in combination with spray-drying, lyophilization or other drying techniques to reduce degradation and/or preserve activity or titer of the enveloped virus prolonging storage, facilitating manufacture, delivery, and use. In certain embodiments, formulations containing enveloped viruses prepared in compositions and methods disclosed herein can be used to produce therapeutics for treating, ameliorating and/or preventing a health condition. In accordance with these embodiments, problems of unstable, rapidly degrading enveloped viruses are averted, reducing production costs and timing where these enveloped viruses are used to create therapeutic products for the healthcare industry.

In certain embodiments, an enveloped virus-containing composition or formulation disclosed herein can include, but is not limited to, one or more enveloped viruses; one or more non-reducing saccharide agents; one or more polysaccharide agents; and at least one amino acid. In certain embodiments, the composition can further include a buffering agent. In some embodiments, the compositions or formulation can further include one or more volatile salts. In some embodiments, the enveloped virus composition is essentially dried using formulations or compositions disclosed herein and methods for drying these formulations or compositions.

In certain embodiments, an enveloped virus-containing composition or formulation disclosed herein is formed by drying an aqueous virus-containing composition or formulation by methods disclosed herein. Prior to drying, the virus-containing composition or formulation can include, but is not limited to, various excipients. In accordance with these embodiments, an enveloped virus-containing composition or formulation prior to drying disclosed herein can include, but is not limited to one or more polysaccharides including, but are not limited to, one or more of hydroxyethyl starch (HES), hydroxypropyl starch, amylose, amylopectin, cellulose, methylcellulose, carboxymethylcellulose, dextrin, cyclodextrin or a combination thereof. In some embodiments, compositions or formulations disclosed herein require HES. In other embodiments, the enveloped virus-containing composition can include one or more non-reducing saccharides including, but not limited to, trehalose, sucrose, lactose, raffinose, gentiarose, melezitose, stachyose, and verbascose or a combination thereof. In some embodiments, the non-reducing disaccharide is trehalose or sucrose. In other embodiments the non-reducing disaccharide is trehalose. In certain embodiments, a formulations for improving stability of an enveloped virus during lyophilization or spray-drying requires trehalose and HES. In some embodiment, at least one amino acid of use in formulations disclosed herein can include, but is not limited to, histidine, methionine, arginine, proline, glycine, isoleucine, leucine, or the like, or other amino acid. In certain embodiments, a formulation or composition disclosed herein requires proline and/or leucine alone or in combination with HES and/or trehalose. In some embodiments, concentrations of components in an aqueous composition or liquid composition or formulation prior to lyophilizing or spray drying can include polysaccharides of about 0.25% to about 7.50%, or about 0.5% to about 5.0% w/v or about 1.0% to about 4.0%; non-reducing disaccharides at a concentration of about 1.0 to about 40.0%; or about 5.0 to about 25.0%, or about 10.0 to about 25.0% w/v, and an amino acid concentration of about 5.0 mM to about 200.0 mM; about 10.0 mM to about 150.0 mM or about 10.0 mM to about 100.0 mM.

In certain embodiments, an enveloped virus-containing composition or formulation disclosed herein is formed by drying an aqueous virus-containing composition or formulation by methods disclosed herein. Prior to drying, the virus-containing composition or formulation can have a pH of about 4.5 to about 9.0, or about 5.0 to about 8.5; or about 5.5 to about 8.0; or about 5.5 to about 7.5; or about 5.5 to about 7.0; or about 6.0 to about 7.0 or about 6.5. In certain embodiments, a formulation or composition disclosed herein requires proline and/or leucine alone or in combination with HES and/or trehalose at a pH of about 5.5 to about 7.5; or about 6.0 to about 7.0; or about 6.5.

In certain embodiments, an enveloped virus-containing composition or formulation disclosed herein is formed by drying an aqueous virus-containing composition or formulation by methods disclosed herein. Prior to drying, the virus-containing composition or formulation can include, but is not limited to, one or more volatile salts including one or more of ammonium acetate, ammonium formate, ammonium carbonate, ammonium bicarbonate, triethylammonium acetate, triethylammonium formate, triethylammonium carbonate, trimethylamine acetate trimethylamine formate, trimethylamine carbonate, pyridinal acetate, and pyridinal formate, or combinations thereof. In certain embodiments, formulations disclosed herein prior to drying can include a volatile salt at a concentration of about 0.5 mM to about 100 mM. In certain embodiments, a volatile salt of use herein can be included in a formulation prior to spray-drying to improve the drying process and to dry an enveloped virus formulation to near completion, improving outcome, stability, and shelf-life longevity, for example. In other embodiments, an enveloped virus-containing composition or formulation disclosed herein does not include a volatile salt. In some embodiments, when lyophilizing a formulation disclosed herein, a formulation does not include a volatile salt. In certain embodiments, an enveloped virus-containing composition or formulation disclosed herein does not include ammonium acetate. In other embodiments, for example, when using a spray drying method disclosed herein, formulations containing one or more enveloped virus can include ammonium acetate.

In certain embodiments, one or more saccharides can be used in formulations disclosed herein at about 0.5% to about 25% (w/v) in an aqueous formulation prior to drying by methods disclosed herein. In other embodiments, one or more polysaccharides of use in formulations disclosed herein can be about 0.01% to about 10.0% (w/v) in a in an aqueous formulation prior to drying by methods disclosed herein. In yet other embodiments, one or more amino acids can be used in formulations disclosed herein at about 5.0 mM to about 100 mM in an aqueous formulation prior to drying by methods disclosed herein. In other embodiments, volatile salts of use in formulations disclosed herein can be about 5.0 mM to about 100 mM in an aqueous formulation prior to drying by methods disclosed herein. In certain embodiment, formulations disclosed herein have a pH of about 4.0 to about 8.0; or about pH 5.5 to about 7.5.

In some embodiments, a spray-dried or lyophilized aqueous formulation contemplated herein can further include an adjuvant before or after lyophilization or spray-drying an enveloped virus where the enveloped virus is part of a vaccine composition for delivering to a subject. In some embodiments, the adjuvant can include an aluminum salt. In other embodiments, the aluminum salt adjuvant of these formulations can include one or more of aluminum hydroxide, aluminum phosphate and aluminum sulfate, or combinations thereof. In other embodiments, the aluminum salt can be in the form of an aluminum hydroxide gel (e.g., ALHYDROGEL™) or other consistency. In certain embodiments, the aluminum salt adjuvant includes aluminum hydroxide.

In certain embodiments, an enveloped virus-containing composition or formulation disclosed herein can be exposed to elevated temperatures where the enveloped virus(es) remains stable with reduced degradation and reduced loss of titer using aqueous formulations/compositions disclosed herein prior to spray-drying or lyophilization. In some embodiments, the enveloped virus-containing composition or formulation disclosed herein can be exposed to elevated temperatures while the enveloped virus-containing composition or formulation is undergoing a drying process such as during spray-dried or similar drying process where the enveloped virus(es) remains stable with reduced degradation. In certain embodiments, a spray-drying or other drying processes used for the enveloped virus formulations disclosed herein can include an inlet temperature of about 400 to about 90° C. with little to no loss of titer or degradation of the enveloped virus. It is noted that given the instability of enveloped viruses to elevated temperatures, it is surprising that the enveloped viruses remain stable at temperatures that would normally degrade the virus in a few seconds to minutes, the enveloped virus in the current formulations under elevated spray drying temperatures remains stable with little to no loss of titer and further can be stored for prolonged periods from about 4° C. to elevated temperatures of about 40° C., or about 35° C. or about 30° C. Therefore, these processes and temperature conditions contemplated herein can be used to rapidly produce an essentially dry, stable enveloped virus formulation with a rapid glassy transition of the formulation for improved manufacturing and reduced product loss. It is contemplated herein that any enveloped virus can be processed using compositions and methods disclosed herein for use in new and existing therapies at reduced cost.

In certain embodiments, an enveloped virus-containing composition or formulation disclosed herein can be exposed to elevated temperatures wherein the enveloped virus(es) remains stable with reduced degradation and reduced loss of titer due to formulation or composition disclosed herein capable of protecting the enveloped virus during the process (e.g., formation of a glassy matrices). In some embodiments, the enveloped virus-containing composition or formulation disclosed herein can be exposed to elevated temperatures during the process by which the enveloped virus-containing composition or formulation is being dried. In accordance with these embodiments, elevated temperatures of exposure for these compositions or formulations during the lyophilization or spray-drying process can include, but are not limited by temperatures ranging from about 40° C. to about 90° C.; 45° C. to about 90° C.; about 50° C. to about 90° C.; about 55° C. to about 90° C.; about 60° C. to about 90° C.; about 65° C. to about 90° C.; about 70° C. to about 90° C.; about 75° C. to about 90° C., or about 88° C. In some embodiments, the enveloped virus-containing composition or formulation disclosed herein can be exposed to elevated temperatures after the enveloped virus-containing composition or formulation is dried such as lyophilized or spray-dried or similar drying process wherein the enveloped virus(es) remains stable with reduced degradation. In accordance with these embodiments, elevated temperatures of exposure for these compositions or formulations after lyophilization or spray-drying or other drying procedure contemplated herein can include but are not limited to, temperatures ranging from about 4° C. to about 100° C., 4° C. to about 80° C.; ° C. to about 60° C.; about 4° C. to about 55° C. about 4° C. to about 50° C.; about 4° C. to about 45° C.; about 4° C. to about 40° C.; about 4° C. to about 35° C.; about 4° C. to about 30° C. or about 4° C. to about 25° C. for at least one hour to several days, for at least one day to several weeks, for at least one week to several months, for about 10 weeks or more depending on the temperature of exposure and conditions for storage. In certain embodiments, enveloped viruses in a liquid formulation other than a formulation disclosed herein degrade within about 1 hour at elevated temperatures, about 50° C. in absence of use of the formulations and drying methods disclosed herein. In other embodiments, enveloped viruses disclosed herein degrade within about a few seconds, a few minutes, about an hour to about a day to about 3 days at elevated temperatures, about 25° C.-100° C., in absence of use of the formulations and drying methods disclosed herein. In other embodiments, enveloped viruses disclosed herein degrade within about a week at elevated temperatures, about 20° C. to less than 40° C., in absence of use of the formulations and drying methods disclosed herein. In some embodiments, an enveloped virus-containing composition or formulation disclosed herein and lyophilized or spray-dried by methods disclosed herein can be exposed to elevated temperatures of about 4° C. to about 100° C.; 4° C. to about 80° C.; ° C. to about 60° C.; about 4° C. to about 55° C. about 4° C. to about 50° C.; about 4° C. to about 45° C.; about 4° C. to about 40° C.; about 4° C. to about 35° C.; about 4° C. to about 30° C. or about 4° C. to about 25° C. for about 1 day to over 2 months or more to up to a year or more, depending on the temperature of exposure and the desired storage, without the need for freezing temperatures and with improved enveloped virus survival at elevated temperatures. In certain embodiments, an enveloped virus-containing composition or formulation disclosed herein and lyophilized or spray-dried by methods disclosed herein can be exposed to elevated temperatures of about 30° C. for at least 10 weeks without degradation of the enveloped virus.

In certain embodiments, an enveloped virus-containing composition or formulation disclosed herein can include one or more enveloped virus(es). In accordance with these embodiments, the one or more enveloped virus(es) can include one or more enveloped DNA viruses, enveloped RNA viruses, or a combination thereof. In certain embodiments, the one or more enveloped DNA virus(es) can include, but are not limited to, baculoviruses, poxviridae, fowlpox virus, herpes viruses, poxviruses, hepadnaviruses, asfarviruses, other enveloped DNA viruses of use in therapeutic applications where formulations and methods disclosed herein can stabilize the enveloped viruses and includes any combination thereof. In certain embodiments, the one or more enveloped RNA virus(es) can include, but are not limited to, lentiviruses, flaviviruses (e.g., dengue, yellow fever, Zika virus), lyssaviruses, ephemeroviruses, and Vesiculovirus, alphaviruses, togaviruses, coronaviruses, hepatitis D, orthomyxoviruses, paramyxoviruses, avian paramyxoviruses rhabdovirus, bunyaviruses, influenza, hepatitis C, measles, cytomegalovirus, Vesicular stomatitis, mumps, and Ebola virus, other filoviruses and a combination thereof or other enveloped RNA viruses. In certain embodiments, enveloped viruses prepared by compositions and methods disclosed herein can be used in the preparation of therapeutics in vitro (for production of a therapeutic outside of the subject for example) or in vivo depending on the use of the particular enveloped virus, where the enveloped virus is administered to a subject for a therapeutic application such as delivery of a therapeutic agent further manipulated within the subject after administration. It is contemplated herein that any use of an enveloped virus in a therapeutic setting will benefit from formulations and processes disclosed herein for production, manufacturing, storage and use.

In certain embodiments, an enveloped virus-containing composition or formulation disclosed herein can include one or more enveloped viruses including, but not limited to, one or more retroviruses. In accordance with these embodiments, the one or more retroviruses can include one or more RNA tumor viruses. In other embodiments, the one or more retroviruses can include, but are not limited to, human immunodeficiency virus (HIV) or other lentiviruses, human T-lymphotropic virus type 1 (HTLV-1), human T-lymphotropic virus type 2 (HTLV-II) or other oncoretrovirus, or other retrovirus known or unknown in the art, forming part of the enveloped virus family.

In certain embodiments, an enveloped virus-containing composition or formulation disclosed herein can include one or more enveloped viruses including, but not limited to, one or more of influenza A virus, influenza B virus, influenza C virus, influenza D virus, rabies, Bunyavirus, herpes virus, dengue virus, respiratory syncytial virus (RSV), corona virus, hepatitis B, hepatitis C, Ebola virus, human cytomegalovirus, lentiviruses or other enveloped virus. In other embodiments, an enveloped virus-containing composition or formulation disclosed herein can include one or more enveloped viruses including, but not limited to, an envelope virus of use in gene therapy or other use such as gamma-retroviruses (e.g., MMSV, MSCV, Sin-RV or others), and any alphavirus. In certain embodiments, compositions and formulations disclosed herein can be used to stabilize enveloped viruses of use in vaccines employing attenuated or killed enveloped viruses or other enveloped viruses use for therapeutic purposes. In certain embodiments, compositions and formulations disclosed herein can be used to stabilize vaccines employing attenuated, killed, or inactivated enveloped viruses. In some embodiments, killed rabies viruses can be stabilized by compositions and methods disclosed herein, leading to vaccine formulations that require minimal or no refrigeration for transport, storage, and delivery to a subject.

In certain embodiments, compositions and formulations disclosed herein can be used to generate CAR-T cells where the enveloped viruses used to produce these cells are stabilized by formulations and processes contemplated herein which in turn provide improved and more efficient outcomes by stabilizing the enveloped viruses used to produce the CAR-T cells for therapeutic purposes. In some embodiments, CAR-T cells can be produced using lentiviruses stabilized by compositions and methods disclosed herein.

In certain embodiments, a pharmaceutical composition including, but not limited to, a composition or formulation disclosed herein is contemplated where the composition or formulation further includes at least one pharmaceutically acceptable excipient. In accordance with these embodiments, a pharmaceutical composition or formulation disclosed herein can include re-constituting or re-hydrating an essentially dried enveloped virus-containing composition or formulation disclosed herein. In certain embodiments, a reconstituted or rehydrated formulation disclosed herein can be used as a prophylactic, for ameliorating a condition, for eliminating an infection or a condition, or for treating a condition disclosed herein. In some embodiments, a reconstituted or rehydrated formulation disclosed herein can be used as a vaccine to reduce onset or prevent a health condition (e.g., infection, cancer or other condition).

Other embodiments, methods are disclosed for creating and using stabilized enveloped virus compositions and formulations disclosed herein. In one embodiment, the method includes, but is not limited to, combining one or more enveloped viruses with a formulation; the formulation including, but not limited to, one or more non-reducing disaccharide agent(s); one or more starches; one or more amino acid; and generating a liquid enveloped virus formulation. In certain embodiments, the enveloped virus composition or formulation can further include one or more volatile salts. In other embodiments, the method can further include freezing the liquid enveloped virus formulation; and lyophilizing the liquid enveloped virus formulation creating an essentially dry powder of an enveloped virus formulation. In other embodiments, the method can further include spray-drying the liquid enveloped virus formulation using an inlet temperature of about 50° C. to about 100° C.; and creating an essentially dry powder of an enveloped virus formulation. In some embodiments, the essentially dry powder of enveloped virus formulation can be stored at elevated temperatures for transport or later use. In yet other embodiments, the method can further include re-constituting or re-hydrating the essentially dried formulation or composition.

Other embodiments, kits for preparing, storing, or delivering formulations and compositions are disclosed herein. In certain embodiments, kits can include at least one container. In other embodiments, kits can include formulations and compositions disclosed herein for making, storing, transporting, and/or using one or more enveloped virus and optionally, a device for delivering the one or more enveloped virus formulation to a subject to prevent, ameliorate, cure, or treat a condition.

In certain embodiments, one or more agents provided to a vaccine formulation using enveloped virus formulations disclosed herein can include, but is not limited to, one or more immunologically-related co-stimulatory agents for improved immune response. In certain aspects, a formulation disclosed herein can be reconstituted to form liquid vaccine formulation. In yet other embodiments, the enveloped virus compositions disclosed herein can go through a glassification step using drying processes disclosed herein to improve viral stability and produce a stable viral product that is an improvement over prior disclosures. In other embodiments, a glassification step can be a rapid transition when using spray-drying processes at elevated temperatures (above 50° C.) in the presence of formulations disclosed herein. In other embodiments, a glass-forming agent (e.g., when freeze-dried the compositions form a glass instead of crystalizing) disclosed herein can include one or more of trehalose, sucrose, or the like or combinations thereof. In some embodiments, the glass-forming agent is present in a weight-to-volume (w/v) concentration of from about 1% to about 25%, or about 5% to about 20%, or about 5% to about 15%, or about 10% or about 9.5% in a liquid formulation prior to lyophilization. In other embodiments, the glass-forming agent can be trehalose present in a concentration of from about 5% to about 15% w/v in the liquid formulation prior to lyophilization. In another embodiment, the glass-forming agent can be trehalose at a concentration of about 9.5% w/v in the liquid formulation or composition prior to lyophilization. Glass-forming agents that can be used in accordance with the various embodiments of the present disclosure can include, but are not limited to, trehalose, sucrose, lactose, raffinose, gentiarose, melezitose stachyose, verbascose and the like.

In certain embodiments, these formulations can be co-lyophilized or spray-dried, stored and/or transported to remote areas where they can be reconstituted with little to essentially no loss of multimeric structure or immunogenicity. In some embodiments, essentially dried formulations disclosed herein can be stored at room temperature for prolonged periods in preparation for use, such as weeks to months at a temperature of about 25° C. to about 40° C.

In some embodiments, a co-stimulatory agent of a reconstituted vaccine formulation of use herein can include one or more agents to improve an immune response. In accordance with these embodiments, one or more co-stimulatory agent can include, but is not limited to, lipid A, lipid A derivatives, monophosphoryl lipid A, chemical analogues of monophosphoryl Lipid A, CpG containing oligonucleotides, TLR-4 agonists, flagellin, flagellins derived from gram negative bacteria, TLR-5 agonists, fragments of flagellins capable of binding to TLR-5 receptors, saponins, analogues of saponins, QS-21, purified saponin fractions, ISCOMS and saponin combinations with sterols and lipids, or combinations thereof. In other embodiments, the co-stimulatory agent can be about 0.05 mg/mL Glycopyranoside lipid A (GLA) or similar agent having similar effects.

In some embodiments, stability of formulations or compositions disclosed herein can be enhanced by the addition of nonionic surfactants. In accordance with these embodiments, surfactants can be added to vaccine or immunogenic formulations at concentrations ranging from approximately 0.1 times the critical micelle concentration of the surfactant in the vaccine composition, to approximately 20 times the critical micelle concentration of the surfactant in the vaccine composition before, during or after lyophilization of the composition. Suitable nonionic surfactants include, but are not limited to, polysorbates for example, polysorbate 80, polysorbate 20, polysorbate 40, polysorbate 60, polysorbate 65 (e.g., Tween 20, Tween 40, Tween 60 and Tween 80), poloxamers for example Poloxamer 188 and Poloxamer 407, Poloxamer 235, Poloxamer 335, Brij, alkylphenol hydroxypolyethylene surfactants such as Triton X100, Triton X114 and Triton X405, and Oligoethylene glycol monoalkyl ethers such as Genapol. In other embodiments, compositions and methods disclosed herein can include urea or urea-like agent (e.g., carbamide peroxide, allantoin, and hydantoin or other similar agent). In yet other embodiments, formulations or compositions disclosed herein can specifically exclude a surfactant. In certain embodiments, formulations or compositions disclosed herein can specifically exclude a hydrophilic nonionic surfactant.

In certain embodiments, compositions, methods and uses for stabilizing multiple enveloped viruses in a single formulations are disclosed. A formulation or application of a formulation that can stabilize multiple enveloped viruses in a mixed virus formulation prior to drying; for example, against degradation or disassembly of a viral structure is contemplated. In certain embodiments, compositions disclosed herein can be used to reduce loss of titer of lyophilized or spray-dried enveloped virus formulations. In other embodiments, compositions disclosed herein can concern a combination of two or more agents (e.g., adjuvant or adjuvant-like agent) provided to formulation where the formulation is then lyophilized or spray-dried.

Embodiments of the present invention provide for novel compositions and methods for a thermally stable enveloped virus formulation. Certain aspects concern partially or fully lyophilizing or freeze-drying the formulation in the presence of a specified formulation. Other embodiments described herein concern lyophilizing enveloped virus formulations to increase stability or decrease degradation or disassembly of the constructs during storage, transportation, delivery resulting in a reduction of product loss and reduction of loss of efficacy.

In certain embodiments, compositions disclosed herein can be frozen on precooled shelves of a lyophilizer and dried under vacuum creating an essentially dry powder formulation. In other embodiments, formulations including trehalose can be lyophilized and dried to prolong shelf-life of the active agents for storage and transport or enhance immunogenicity.

Suitable vectors for cloning and expressing polypeptides for transfecting enveloped viruses disclosed herein of the present invention are well known in the art and commercially available. Further, suitable regulatory sequences for achieving cloning and expression, e.g., promoters, polyadenylation sequences, enhancers and selectable markers are also well known. The selection of appropriate sequences for obtaining recoverable protein yields is routine to one skilled in the art.

Other embodiments can include polynucleotides that encode chimeric proteins and complexes/capsomeres of use to transfect enveloped viruses disclosed herein to create therapeutic agents contemplated herein. Accordingly, any nucleic acid sequence, which encodes the amino acid sequence of chimeric proteins and complexes/capsomeres, can be used to generate recombinant molecules that express chimeric proteins and complexes/capsomeres. It will be appreciated by those skilled in the art based on the present disclosure that as a result of the degeneracy of the genetic code, a multitude of nucleotide sequences encoding chimeric proteins and complexes/capsomeres of the present disclosure, some bearing minimal homology to the nucleotide sequences of any known and naturally occurring gene, may be produced. Thus, the disclosure contemplates each, and every possible variation of nucleotide sequence that could be made by selecting combinations based on possible codon choices. These combinations are made in accordance with the standard triplet genetic code as applied to the nucleotide sequence of naturally occurring chimeric proteins and complexes/capsomeres of the present disclosure, and all such variations are to be considered as being disclosed.

Chimeric proteins have application in both prophylactic and therapeutic vaccines and diagnostics. The suitability of the chimeric proteins and capsomeres produced for use as vaccines or as diagnostic agents can be confirmed by reaction with antibodies or monoclonal antibodies which react or recognize conformational epitopes present on the intact vision and based on their ability to elicit the production of neutralizing antiserum. Suitable assays for determining whether neutralizing antibodies are produced are known to those skilled in the art based on the present disclosure.

Certain embodiments disclosed herein can include kits of use for storage and transport of one or more essentially dried enveloped protein formulation disclosed herein, or one or more container disclosed herein is contemplated. In certain embodiments, kits contemplated herein can be kits able to withstand elevated temperatures and/or low temperatures, for use at temperature-ranges as disclosed herein (e.g., 4° C. to about 60° C.). In accordance with these embodiments, a kit can include a container having one or more lyophilized or spray-dried envelope virus formulation disclosed herein.

As disclosed herein, spray-drying and lyophilization are widely used to enhance the storage stability of biologics and reduce cold chain dependence but the fragility of enveloped viruses has made these technologies challenging until the instant disclosure. In some embodiments, trehalose was analyzed in formulations disclosed herein due to its ability to form an amorphous glassy matrix during drying and for its stabilizing ability of proteins and lipid membranes during desiccation. An amorphous state has been shown to be advantageous compared to crystalline states for storage of other viruses so the instantly claimed compositions and methods were sought to seek enveloped viruses in a similar state. It is understood by these inventions disclosed herein that biomolecules embedded in the amorphous matrix trehalose formed during drying experience restrict molecular mobility, significantly decreasing the rates of physical and chemical degradation pathways. In addition, low molecular weight additives which plasticize the glassy matrices have also been demonstrated to enhance protein stability. It was discovered herein that volatile salts can act as a plasticizer (e.g., ammonium acetate) under the current conditions as well as reduce drying times without increasing residual salt. In other embodiments, polysaccharide additions to formulations disclosed herein were thought to lower the Tg of a dried formulation (e.g., HES. Polysaccharides of use herein are also thought to confer stability to proteins and lipids in the dry state through hydrogen bonding and resulting associated steric restrictions. In some embodiments, enveloped virus formulations disclosed herein demonstrated enhanced thermal stability with little to no titer loss during storage for 10 weeks at temperatures between 4 and 40° C. These inventions demonstrated significant enhancement in the stability of enveloped viruses when embedded in a glassy matrix compared to a liquid suspension or drying processes not using the claimed formulations. It was observed herein that titer retention during storage was also enhanced compared to existing lyophilized live viral vaccines, which still require refrigerated or frozen storage. Formulated enveloped viruses disclosed herein demonstrated enhanced stability in liquid suspension at 4° C. compared to enveloped viruses not in these formulations in liquid suspension at 4° C. In other embodiments, a dried enveloped virus preparation disclosed herein demonstrated almost no titer loss during acute incubations up to 85° C., while liquid formulations exhibited over three logs of titer loss when incubated above 50° C. for the same amount of time. It is hypothesized that enveloped virus behavior under these conditions likely have at least two structural components that participate in viral inactivation reactions, each with associated different kinetics. These inactivation kinetics are important to viral function because all structural components of the enveloped virus (surface proteins, lipid envelope, genetic material, etc.) must remain intact prior to infection, but then these same structural components of the enveloped virus must dissociate after entering a host cell. Therefore, integrity of a lipid envelope of an enveloped virus is critical for entry into and infection of host cells for nearly all enveloped viruses. It is thought that in a dried state, saccharides as disclosed herein (e.g., trehalose and sucrose) can interact with lipids and maintain the structure of membranes and lipid bilayers and vesicles during drying or freezing.

As disclosed herein, both spray-drying and lyophilization significantly enhanced the stability of enveloped viruses over long periods of time at moderate temperatures, and for over one hour at temperatures approaching the Tg's (e.g., up to 85° C.) of the dried formulations. In some embodiments, it was demonstrated that enveloped viruses within spray-dried formulations appeared to have slightly better stability than in the lyophilized preparations. Differences in the glasses formed during each process may account for these subtle differences in stability. Higher Tg's were measured in the spray-dried preparations (See FIG. 9). Additionally, drying and glass formation occur over different timescales in spray-drying and lyophilization. In the spray dryer, drying of a droplet occurs over fractions of a second, whereas primary drying in lyophilization typically takes over roughly 10 hours, or about 4 orders of magnitude slower. It is considered that even in the same formulation, the glass formed during these two processes likely differs in residual stress, relaxation kinetics, specific surface area, and mobility but both processes provided highly stable enveloped viruses using the disclosed formulation herein.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

It has been observed that enveloped viruses are very fragile and unstable at elevated temperatures. To use stabilizing processes to dry these viruses has been a challenge due to the challenges of these drying processes themselves. It is thought that hot drying gas temperatures encountered during drying processes have been suggested to damage virus particles during spray-drying. Generally, drying gas in the drying chamber of a spray dryer is well mixed. In this environment, the temperature at the surface of the droplet will first increase to the wet-bulb (Twb) temperature while solvent evaporation is limited by the energy required for evaporation. Eventually, solute concentration at the droplet surface will increase, leading to crust formation and a decreased drying rate. Beyond this point, drying particles will reach at most the dry-bulb temperature of the surrounding gas, which will have cooled substantially compared to the inlet gas temperature due to evaporative cooling. Thus, during a drying process of an enveloped virus contemplated herein, the suspended droplets containing virus particles will experience temperatures that rise only to the wet bulb temperature during early stages of drying, and only start to approach the outlet gas temperature after significant drying has occurred making it a possible stabilizing option if initial formulation conditions are conducive to stabilizing the virus during these transitions. At this point, evaporation will have resulted in concentration of the formulation excipients, resulting in formation of a protective glassy matrix. Previous studies indicated that titer loss of a vaccine candidate correlated roughly with increasing dry gas outlet temperature, where a higher outlet temperature increased titer loss. In the instant studies, no significant difference was observed between drying conditions but given the rapid drying of enveloped virus formulations disclosed herein and more reasonable costs of spray-drying, this method may be used over lyophilization techniques. In these experiments and as contemplated herein, the drying gas was dehumidified prior to entering the drying chamber, and the mass of water entering via the formulation is low compared to the mass of dry air entering the chamber, resulting in a relative humidity (RH) in the spray dryer below approximately 5%. For inlet gas temperatures of 88° C. and 50° C., the measured outlet temperatures were approximately 60° C. and 35° C., respectively. At this RH and these outlet gas temperatures, the Twb were approximately 23° C. or approximately 14° C., respectively, as determined using a psychrometric chart. Therefore, as disclosed herein, while virus-containing droplets were in a liquid state, evaporative cooling limited the temperature to which viruses were exposed, whereas exposure to the highest temperatures present in the spray dryer occurred only when the viruses were encased in a protective glassy matrix.

It is thought that drying may also impose mechanical stresses such as shear stress during atomization, exposure to air-water interfaces, and hyper-osmotic stress due to solute concentration. These stresses have also been hypothesized to cause titer loss during spray-drying of viruses in previous studies, although the effects of these stresses on enveloped viruses have not been well isolated or systematically evaluated in the literature. As hypothesized herein and previously observed, osmotic pressures exerted on capsids or the viral envelope due to drying-induced concentration of solutes during freezing or drying was likely to damage enveloped viruses but surprisingly, no titer loss due to this drying step was observed under disclosed conditions herein. Notably, during spray-drying, water removal and solute concentration occur rapidly (e.g., on a timescale on the order of 100 ms). It is thought that during spray-drying, enveloped virus particles rapidly become trapped in the formulations disclosed herein of a viscous, glassy matrix, preventing damage from occurring before the increasing osmotic pressure causes significant virus particle deformation. Surprisingly, it is thought that rapid viscosity increases due to disaccharides and polysaccharides during drying likely provided enveloped viruses protection against drying-induced increases in osmotic pressures. Together, using a relatively low drying gas temperature, rapid drying rates and high Tg formulation explain why little loss of viral titer during drying was observed. Additionally, the effects of osmotic stress on viruses during lyophilization have not been identified, though they have been observed in isolation. The slow rate of drying during lyophilization might allow for water and salt ions to move across the viral envelope to compensate for osmotic pressure changes. This, along with the same increase in viscosity due to the excipients provided herein, might also explain why increased osmotic pressure occurring during lyophilization did not affect viral titers.

### Example 1

### Physical Characterization of Spray Dried Baculovirus

In one exemplary method, an exemplary enveloped virus, baculovirus (BV) was spray dried at two drying conditions: an inlet temperature of 88 degrees Celsius (° C.) or 50° C. Following spray drying, the glass transition temperature (Tg) of spray dried baculovirus was measured by modulated differential scanning calorimetry (MDSC). Both samples had a T_{g} of approximately 110° C. (as illustrated in FIG. 1A), indicating a very low (<2%) moisture content for a trehalose-based glass. Spray dried particles at each drying condition had similar particle size and morphology as measured by flow imaging microscopy (as illustrated in FIG. 1B) and scanning electron microscopy (SEM) (as illustrated in FIG. 1C for the particles dried at an inlet temperature of 88° C. and in FIG. 1D for the particles dried at an inlet temperature of 50° C.).

### Example 2

### Storage Stability of Spray Dried and Lyophilized Baculovirus

In another exemplary method, liquid, spray dried, and lyophilized baculovirus were incubated at 4° C.-40° C. for up to 10 weeks. Periodically, titers of liquid and dried samples were measured. Log loss titer was calculated compared to the titer before drying

$$( log loss titer = {log}_{10} ( \frac{titer before drying}{sample titer} ) ) .$$

When no activity was detected by the titer assay, a value of 1.0 was used as the sample titer for LLT calculation. Unformulated viral stock (2.3E+08 pfu/mL) was measured as a control. For all samples, measurements were not taken after at least two consecutive measurements with no activity.

Unformulated (as illustrated in FIG. 2A) or formulated (as illustrated in FIG. 2B) baculovirus incubated at 40° C. lost all activity in one week or less. At 25° C. or 30° C., unformulated viral stock (as illustrated in FIG. 2A) lost over 2 log of activity over 10 weeks. Baculovirus spray dried at two different drying conditions showed no activity loss upon spray drying (day 0) and no change in activity over 10 weeks after incubation at 4° C.-40° C. (as illustrated in FIGS. 2C-2D).

In FIGS. 2A-2D, n=3, the error bars represent standard deviation of calculated log loss titer for each replicate. The initial titer of viral stock was 2.3E+08 pfu/mL. The initial titer of liquid formulated, and spray dried samples were 1.2E+06 pfu/mL.

Preliminary stability data for lyophilized baculovirus in the same formulation demonstrates a moderate titer loss upon lyophilization (day 0), and up to 0.5 log loss titer over a 10-week incubation at either 4° C. or 25° C. incubation (as illustrated in FIG. 3, N=1).

### Example 3

### Acute Stability of Liquid, Spray Dried, and Lyophilized Baculovirus

Formulated liquid and spray dried and lyophilized baculovirus was incubated for 1 hour at 25° C.-97° C. Log loss titer vs. titer of the same sample held at 4° C. for 1 hour was calculated. Formulated liquid baculovirus showed over 2 log activity loss over 1 hour incubation above 40° C. (as illustrated in FIG. 4). Spray dried and lyophilized baculovirus showed no titer loss up to 97° C. (as illustrated in FIG. 4). In FIG. 4, n=1.

As illustrated in exemplary FIG. 4, log loss titer of formulated, spray-dried, and lyophilized BV incubated for one hour at various temperatures were analyzed up to 100° C. BV in liquid formulation (purple squares) demonstrated highly significant titer loss when stored above 40° C. for a little as one hour. In contrast, spray-dried (green triangles) and lyophilized (light blue circles) of BV demonstrated no titer loss up to 85° C. (n=3) in formulations disclosed herein.

### Example 4

### Lyophilization Using Various Excipients

In another exemplary method, various excipients were tested to assess the contribution of these components regarding stabilizing the exemplary enveloped virus. For example, FIG. 5 illustrates the titer loss on a log scale for 11 different formulations after the lyophilization process (vs. before lyophilization or just in liquid formulations). Notably, when starch is not included (HES), a high titer loss was observed for the lyophilization process. The formulations included the following exemplary formulations:
S1: 9.5% trehalose, 2.5% HES, 10 mM 1-histidine, 40 mM ammonium acetate, pH 6.5 (matching what we used in spray drying);
S2: 9.5% trehalose, 10 mM 1-histidine, 40 mM ammonium acetate (AA), pH 6.5;
S3: 9.5% trehalose, 2.5% HES, 10 mM 1-histidine, pH 6.5;
S4: 9.5% trehalose, 5.0% HES, 10 mM 1-histidine, 40 mM ammonium acetate (AA), pH 6.5;
S5: 9.5% trehalose, 2.5% HES, 10 mM 1-histidine, 40 mM ammonium acetate, 25 mM proline, pH 6.5;
S6: 9.5% trehalose, 2.5% HES, 10 mM 1-histidine, 40 mM ammonium acetate, 1 mg/mL leucine: pH 6.5;
S7: 9.5% trehalose, 10 mM 1-histidine, 40 mM ammonium acetate, 1 mg/mL leucine pH 6.5;
S8: 9.5% trehalose, 2.5% HES, 10 mM 1-histidine, 40 mM ammonium acetate, 230 mM arginine, pH 6.5;
S9: 9.5% trehalose, 2.5% HES, 10 mM 1-histidine, 40 mM ammonium acetate, 1.2 uM polysorbate 80, pH 6.5;
S10: 9.5% trehalose, 2.5% HES, 10 mM 1-histidine, 40 mM ammonium acetate, 120 uM polysorbate 80, pH 6.5; and
S11: 9.5% trehalose, 2.5% HES, 10 mM 1-histidine, 40 mM ammonium acetate, 0.4 M urea, pH 6.5

In another example, selected formulations were exposed to 2 temperatures and incubated for prolonged periods to assess degradation of the tested enveloped virus. For example, FIG. 6 illustrates preliminary stability data for lyophilized BV of 10 example formulations. Testing was not continued with formulation S2 (no HES) as it had no activity after lyophilization in this test. BV in the other 10 formulations were lyophilized and a similar stability experiment (similar to what is illustrated for the spray dried powder) was performed and is illustrated as yellow/squares are 25° C. and blue/circles are 4° C.

### Example 5

### Stability of Another Enveloped Virus

In another exemplary method, various excipients based on previous experiments indicated above were used to test stability of inactivated rabies virus (enveloped virus) under spraying dried from a liquid composition in a formulation of 9.5% trehalose, 2.5% hydroxyethyl starch (HES), 50 mM ammonium acetate, 10 mM histidine. Spray dried powders were incubated for about 3 months to assess stability at various temperatures including at 25° C. (yellow/circles), 40° C. (blue, upright triangle), or 50° C. (green, upside-down triangle) prior to reconstitution and injection. Reconstituted spray dried powders at a concentration of 0.04 IU/dose were injected on days 0, 7, 21 to assess viral titer and enveloped virus survival of the rabies virus formulation. Control: Single injections on Day 0 of Rabivax-S liquid at 0.09 IU/dose. Rabivax S liquid was stored at 4° C. No loss of immunogenic activity of rabies virus was observed after 3-month storage at 25, 40, or 50° C. Immunogenic activity was unaffected by spray drying and in fact was stabilized even at temperatures of 50° C. (See FIG. 7) Example 6

### Biophysical Characterization of Spray-Dried and Lyophilized BV

In another example as illustrated in FIG. 8, XRD data demonstrated that the spray-dried powders and lyophilized cakes were fully amorphous (no sharp Bragg peaks), while unprocessed trehalose was highly crystalline. Peaks observed for unprocessed trehalose were consistent with those reported in the literature, showing Bragg peaks at 20 values of 15.2° and 23.8°. Tg of the amorphous spray-dried and lyophilized powders was measured by DSC. Placebo formulations (containing 9.5% trehalose, 2.5% HES, 10 mM 1-histidine, 40 mM ammonium acetate, pH 6.5 but without BV) spray-dried at either Tin (88 or 50° C.) exhibited Tg values of approximately 82° C., while placebo lyophilized cakes had a Tg of 72° C. Previously, using Karl Fischer titration, it was found that spray-dried powders in this and similar formulations have less than 2.0% residual water, and lyophilized cakes of the same formulation have less than 0.5% residual water (data not shown). All Tg values were lower than expected for a dry trehalose glass, likely due to the inclusion of HES and other low molecular weight additives. Notably, the Tg of the spray-dried powders was higher than temperatures of the outlet drying gas for all conditions tested.

FIG. 8, represents XRD spectra of spray-dried and lyophilized placebo samples and unprocessed trehalose were examined. Unprocessed trehalose (n=1 scan) is highly crystalline and shows agreement with the literature for characteristic peaks of trehalose dihydrate. Spray-dried (SD) placebo powder was examined using heated air at an inlet temperature (Tin) of 88° C. or 50° C., and lyophilized placebo cakes (n=2-4 scans) were observed to be highly amorphous, indicating that the disaccharide (e.g., trehalose) was in a glassy state following lyophilization or spray-drying, and independent of the inlet temperature of the gas used in spray-drying.

### Example 7

In another example size and morphology of spray-dried powders were analyzed containing BV by FIM (FIG. 9) and SEM (FIG. 10). Particles were prepared by spray-drying into heated air at two different temperatures (88 or 50° C., as measured at the inlet to the spray dryer). The resulting particle size distributions as measured by FIM were unaffected by the temperature of the inlet drying gas (FIG. 9). SEM revealed that the spray-dried particles had a dimpled morphology (FIG. 10). Additionally, the SEM was consistent with the FIM, demonstrating that both spray-drying conditions used in this study resulted in particles with similar size, in addition to their similar morphology (FIG. 10). As illustrated in FIG. 9, the table represents measured glass transition temperatures Tg of placebo spray-dried powders and lyophilized cakes. The listed Tg is the average of two or more replicates with the standard deviation given in parentheses. Placebo powder was spray-dried using heated air at an inlet temperature (Tin) of 88 or 50° C. Illustrated in FIG. 10 is a bar graph depicting particle size distribution of spray-dried particles measured by FIM. Particle size distributions for particles formed by spray-drying using heated air with a temperature of 88° C. (blue bars) or 50° C. (orange bars) were indistinguishable. Size distribution of the particles was estimated using the ESD values returned by VisualSpreadsheet for each particle.

### Example 8

In another example BV titers were measured immediately after formulation, spray-drying, and lyophilization. No titer loss occurred between formulation and spray-drying or lyophilization (FIG. 11). Titers before and after drying were not significantly different by a two-sample t-Test (p-value >0.05). As illustrated in FIG. 11, titer of BV in liquid formulations prior to drying (blue bars/left) and in reconstituted lyophilized or spray-dried formulations (green bars/right) are presented. No loss of titer occurred during spray-drying BV at drying gas inlet temperatures of 88° C. (n=7) or 50° C. (n=3) or during lyophilization (n=3). Titers before (blue bars/left) and after (green bars/right) drying were not significantly different by a two-sample t-Test (p-value >0.05).

### Example 9

### Storage Stability of Spray-Dried and Lyophilized BV

In another example, BV was stored for up to 10 weeks at 4, 25, 30, or 40° C. (all temperatures were substantially below the Tg (FIG. 9) as supplied in Sf900-III SFM (viral stock), in liquid formulation, or after spray-drying or lyophilization in the same formulation. Spray-dried powders and lyophilized cakes were stored in sealed vials backfilled with dry nitrogen. Titer was measured periodically over 10 weeks by quantifying the number of sf9 cells infected by flow cytometry for a given sample. Log loss titer of BV during storage is shown over time (FIGS. 12A-12B). BV in the stock solution and in the liquid formulation was stable at 4° C. for 10 weeks, but rapidly lost activity when stored at 25° C. or above (FIG. 12A-12B). Notably, in both types of liquid samples, when stored at 40° C., BV lost nearly all detectable activity in less than one week. In contrast, when spray-dried BV was incubated at temperatures from 4-30° C., surprisingly no loss in activity during storage was observed over 10 weeks (FIGS. 12C-12D). After 10 weeks of incubation at 40° C., BV spray-dried at drying gas temperatures of 88 and 50° C. demonstrated less than 0.1 and 0.5 log loss titers, respectively (FIGS. 12C-12D). Lyophilization also improved the storage stability of BV, with less than 0.25 log loss titer after incubation at 25° C., and less than 1.0 log loss titer after incubation for 10 weeks at 40° C. (FIG. 12E).

FIGS. 12A-12E illustrate exemplary graphs of log loss titers for BV during storage in liquid, spray-dried powders, and lyophilized cakes. In these experiments, BV was incubated at 4° C. (blue squares), 25° C. (green circles), 30° C. (orange triangles) or 40° C. (red diamonds) as supplied (viral stock, A), after formulation added to stock (B), after spray-drying at a Tin of 88° C. (C) or 50° C. (D), and after lyophilization (E). Spray-dried and lyophilized BV demonstrated enhanced storage stability at 25° C. and above (40° C.) compared to liquid stock and formulated BV (n=3). nd=not determined (measurements were not repeated after samples showed no activity on two consecutive days). Therefore, formulations disclosed herein can provide stability to enveloped viruses during spray-drying or lyophilization for prolonged shelf-life and storage, creating more reliable stocks for use in therapeutic indications.

In another exemplary method, to further probe BV thermal stability, formulated, spray-dried, and lyophilized BV were incubated for one hour at various temperatures from 4-97° C. (FIG. 4). The observed difference in stability of BV in the liquid and dried form is apparent at temperatures above 40° C. After one hour, BV in liquid formulation lost over 3 log titer when incubated above 40° C. (FIG. 4). In contrast, spray-dried and lyophilized BV demonstrated almost no titer loss when stored for one hour at up to 85° C. (FIG. 4). Surprisingly, it was observed that only after incubation at 97° C. (which is nearly 10° C. above the Tg of the spray-dried and lyophilized samples (FIG. 9) for one hour did the spray-dried and lyophilized BV begin to demonstrate activity loss, but only up to 0.5 log loss titer.

### Example 10

In another example, scanning electron microscopy (SEM) of lyophilized placebo formulations are illustrated in an exemplary image (FIG. 13). Cross-sections of lyophilized placebo cakes were mounted on imaging stubs via double-sided carbon tape for this process.

### Materials and Methods

### Reagents

Trehalose was purchased from Pfanstiehl (Waukegan, Illinois). All buffer salts were of reagent grade or higher. Ammonium acetate was from J. T. Baker (Avantor, Radnor, PA). L-histidine was from Sigma-Aldrich (St. Louis, MO). HES (Hetastarch) was purchased from McKesson (Irvine, TX) and was exchanged into 10 mM 1-histidine by tangential flow filtration. Sf900-III serum free media (SFM), gentamicin, amphotericin B, and propidium iodide (1.0 mg/mL in water) were purchased from ThermoFischer Scientific, USA.

### Viral Stock

Recombinant baculovirus (BV) encoding green fluorescent protein (GFP) was purchased. BV working stock was produced from the initial stock in sf9 cells. BV working stock was suspended in Sf-900 III SFM from Thermo Fischer Scientific at approximately 2.3E+08 pfu/mL.

### Sf9 Cell Culture

Sf9 cells were grown in baffled shaker flasks in Sf-900 III SFM. Cells were cultured in a shaker-incubator at 27° C., shaking at 130 rpm with no light. Cells were passaged every 2-3 days to a concentration of 8.0E+05-1.2E+06 cells/mL.

### Exemplary Formulation

Formulation 1: 9.5% trehalose, 2.5% hydroxyethyl starch (HES), 10 mM 1-histidine, 40 mM ammonium acetate, pH 6.5. BV was formulated by dilution from working stock (approximately 2.3E+08 pfu/mL) to between 1.0E+06 pfu/mL-3.0E+06 pfu/mL.

HES in 0.9% NaCl was exchanged into 10 mM 1-histidine by tangential flow filtration using a VivaFlow 50R 10 k MWCO membrane.

### Spray Drying

An exemplary enveloped virus BV was used in these studies. BV stock was diluted to approximately 1.2E+06 pfu/mL in about 9.5% trehalose, about 2.5% HES, about 10 mM 1-histidine, about 40 mM ammonium acetate, at about pH 6.5 (“exemplary formulation”). Formulated BV was spray-dried on the same day that formulations were prepared using a Buchi B-290 spray dryer and inline dehumidifier B296 (New Castle DE) in a closed-loop configuration. To prevent pressure from building up in the spray-dryer with a closed-loop configuration, air- and water-outlet streams from the dehumidifier were bubbled into a 10% bleach solution. Dehumidified air was used as the drying gas. Two drying gas temperatures were used, 88° C. or 50° C., at a constant air flowrate of 32 m³/h, which resulted in outlet temperatures of 57-60° C. or 34-37° C., respectively. For both drying gas conditions, nitrogen was used as the atomizing gas at a flowrate of 414 L/h and the formulation was pumped to the spray dryer at a feed rate of 1.0 mL/min. A two-fluid atomizing nozzle with an outlet diameter of 0.7 mm was used for atomization. After spray-drying, powders were placed in 5-mL vials and loaded into a LyoStar II lyophilizer from FTS Systems (Warminster, PA) with the shelf temperature set to 4° C. The chamber was evacuated of air to a pressure of 60 mTorr, then backfilled with dry nitrogen, after which time vials were capped allowing the dried powders to be stored in a dry environment until characterization. For assays, spray-dried powders were reconstituted to their original volume in sterile deionized (MilliQ®) water. Placebo powder was prepared in the same manner for physical characterization, but BV was omitted from the formulation. Upon formulation, the added liquid suspension of BV in Sf-900 III SFM comprised approximately 0.5% (by volume) of the total solution, so its omission did not significantly change the physical properties of the powders. Tg determination by modulated differential scanning calorimetry (MDSC)

Glass transition temperature (Tg) of spray dried and lyophilized samples was measured by MDSC. 3-10 mg of sample was loaded into a hermetically sealed aluminum pan. Samples were heated from about 25° C. to about 140° C. at a rate of about 3° C./min with an amplitude of about 1° C./min and a period of about 60/s. Measurements were performed in triplicate (3 sample pans) for each batch of spray dried and lyophilized material. Reversing, non-reversing, and total heat flow were recorded. To determine the glass transition temperature (Tg) using OriginPro, the average of the first derivative of the reversing heat flow (after removing duplicates by averaging the y-values at repeated x-values and smoothing data by a Lowess algorithm) was plotted versus temperature. Following baseline subtraction, peaks were identified in OriginPro.

### Lyophilization

BV was formulated as described for spray-drying. Within a day after BV was formulated, 1.0 mL aliquots of formulated BV were filled into 3-mL Type II glass vials (Schott, Mainz, Germany) with 13-mm rubber butyl stoppers (DWK, Wertheim, Germany) inserted halfway. Formulated BV was lyophilized in a LyoStar II lyophilizer from FTS Systems. Vials were loaded onto pre-cooled lyophilizer shelves at −1° C. and surrounded by a ring of unstoppered vials containing 1.0 mL of water to minimize radiative heat transfer effects from lyophilizer walls. To initiate freezing, the shelf temperature was decreased to −40° C. at an average rate of 2° C./min. Primary drying was initiated by decreasing the pressure to 60 mTorr before increasing the temperature to −20° C. at a rate of 1.0° C./min. This temperature was maintained for 20 hours, a time nearly twice that estimated to be required for primary drying, based on preliminary drying rate data extrapolated to the estimated moisture content at the glass transition temperature for the maximally freeze-concentrated solid (data not shown). For secondary drying, the temperature was raised to 30° C. at 0.5° C./min. then held at 30° C. for 5 hours. Finally, the chamber was backfilled with dry nitrogen to atmospheric pressure, at which time vials were fully stoppered and crimped. For assays, lyophilized samples were reconstituted to their original volume in sterile MilliQ® water. Lyophilized placebo samples without added BV were prepared in the same manner and used for physical characterization.

### X-ray Diffraction Studies

X-ray powder diffraction (XRD) patterns of placebo spray-dried powders, placebo lyophilized cakes, and unprocessed trehalose were recorded via a Rigaku SmartLab 9 kW X-ray diffractometer (Ceder Park, TX). Each sample type was pressed, using a glass slide, into a channel that had been etched into a silicon wafer. Unprocessed trehalose was ground in a mortar and pestle before loading. Samples were subjected to Cu K-alpha (wavelength of 0.154 nm) radiation for 10 min. per scan, for up to four scans each. Intensities of scattered X-rays were recorded at angles ranging from −90° to +900 at a rate of 10°/min.

### Differential Scanning Calorimetry

A TA Discovery DSC 2500 (TA, New Castle, DE) was used for T_{g} measurements. Approximately 2-8 mg of spray-dried or lyophilized sample was packed into aluminum pans and hermetically sealed. Sample pans were heated from 25° C. to 140° C., cooled back to 25° C., then heated once more to 140° C., all at a rate of 25° C./min. The T_{g} was identified as the maximum peak in the first derivative of the second heating scan. The average T_{g} is reported from two or three replicates.

### Flow Imaging Microscopy (FIM)

Particle size distributions were measured by flow imaging microscopy (FIM) using a FlowCam VS (Fluid Imaging Technologies, Inc., Scarborough, ME). Approximately 1 mg of spray-dried powder was resuspended in dry 200-proof ethanol and dispersed by sonication for 1 min. Dry ethanol was stored with 3× molecular sieves to ensure no water was present which would dissolve trehalose or HES. Powder suspensions were further diluted in dry ethanol to achieve a concentration between 300,000-1,000,000 particles/mL as counted by the FlowCam. 150 μL of the diluted suspension was loaded into the FlowCam per run. Each spray-dried powder was analyzed in triplicate. Particle sizes were determined using the estimated spherical diameter (ESD) returned by VisualSpreadsheet for each particle.

### Scanning Electron Microscopy (SEM)

Dry powders were mounted on imaging stubs using adhesive carbon tape, and sputter coated with platinum to a thickness of approximately 3 nm using a 108 Auto Sputter Coater from Cressington (Liverpool, UK). Stubs were imaged on a Hitachi SU3500 Variable Pressure scanning electron microscope (SEM) (Hitachi, Tokyo, Japan).

### BV Activity Measurements (Titer)

Unformulated BV stock (control) was diluted to an MOI of 0.001-1.0 in Sf-900 III for an 8-point standard curve during each experiment. BV at an MOI of 0.1 and 0 were used as plate controls. Spray dried and lyophilized samples were reconstituted in sterile DI water at their original volume. 150 uL sample and standards were added to two or one wells, respectively, containing sf9 cells in Sf-900 III SFM at a final concentration of 1.2E+06 cells/mL. Mini suspension cultures in 12-well plates were incubated for 48 hours at 27 C, shaking at 130 rpm with no light. After 48 hours, 1.0 mL cell suspensions from each well were harvested and washed twice by spinning them down in a deep well plate and resuspending the cells in fresh media. On the final wash, propidium iodide (PI) was added to the cell suspensions at 5 uL/mL. Cells sat for 15-30 minutes following addition of PI. Cells were transferred to tubes or a clear 96 well plate. Cells were analyzed by flow cytometry using a BD Celesta. 10,000 cells were recorded in triplicate for each sample.

### Titer Assays and Flow Cytometry

For titer assays, sf9 cells were washed once and resuspended in Sf-900 III SFM with 10 μg/mL gentamicin and 0.25 μg/mL amphotericin B. Cells were seeded at 1.2E+06 cells/mL in 12-well plates. 150 μL of liquid or reconstituted samples were added to bring the total well volume to 1.5 mL. Titers were determined in duplicate wells for samples, whereas standards were analyzed in single wells. Plate standards at an MOI of 0.1 or 0 were included on each plate as a positive and negative control. 48-h post infection, sf9 cells were harvested and washed twice by centrifugation with fresh media. To each well, 5 μL of propidium iodide stain were added per mL of cell suspension. Stained cell suspensions were incubated at least 15 min. at room temperature before analysis of the cells by flow cytometry.

Cell suspensions were analyzed on a BD FACS Celesta (BD BioSciences, Franklin Lakes, NJ) and the data were analyzed in FlowJo (v10) software. Live cells were identified by propidium iodide staining, and single, live cells were identified by forward and side light scatter. Cells infected by GFP-encoding BV expressed GFP in a dose-dependent manner and were identified by 488/530 laser intensity. The fraction of infected, live, single cells out of all live, single cells was used to calculate the titer.

### Exemplary Titer Analysis

Dead cells were excluded from the analysis based on PI signal. Single cells were identified by FSC-W vs. FSC-A. Live single cells expressing GFP were identified by signal at 488/530 nm. The fraction of live single cells expressing GFP was used to back-calculate sample MOI based on the average of the standard curve over multiple experiments. Titer was calculated by multiplying the MOI by the initial cell concentration used in the titer assay.

### Statistical Analysis

Statistical analyses were performed in Origin Pro (2021). A Grubbs outlier test was used to identify outliers using Origin Pro (2021). A cut-off of 0.05 was used to determine significance. A two-sample t-Test with a significance level of 0.05 was used to compare the mean titers before and after spray-drying or lyophilization.

All of the COMPOSITIONS and METHODS disclosed and claimed herein can be made and executed without undue experimentation in light of the present disclosure. While the compositions and methods have been described in terms of particular embodiments, it is apparent to those of skill in the art that variations maybe applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. An enveloped virus-containing composition comprising:
one or more enveloped viruses;
one or more non-reducing saccharide agents;
one or more polysaccharides,
one or more amino acids; and
a buffer.

2. The enveloped virus-containing composition according to claim 1, wherein the enveloped virus composition is essentially dried.

3. (canceled)

4. The enveloped virus-containing composition according to claim 1, wherein the one or more polysaccharides comprise one or more of hydroxyethyl starch (HES), hydroxypropyl starch, amylose, amylopectin, cellulose, methylcellulose, carboxymethylcellulose, dextrin, cyclodextrin or a combination thereof.

5. The enveloped virus-containing composition according to claim 1, wherein the one or more non-reducing saccharides comprises one or more of trehalose, sucrose, lactose, raffinose, gentiarose, melezitose, stachyose, verbascose, or a combination thereof.

6. The enveloped virus-containing composition according to claim 1, wherein the composition comprises one or more volatile salts and the one or more volatile salts comprise one or more of ammonium acetate, ammonium formate, ammonium carbonate, ammonium bicarbonate, triethylammonium acetate, triethylammonium formate, triethylammonium carbonate, trimethylamine acetate trimethylamine formate, trimethylamine carbonate, pyridinal acetate and pyridinal formate, or combinations thereof.

7. The enveloped virus-containing composition according to claim 2, wherein the essentially dried enveloped virus-containing composition has been exposed to elevated temperatures of about 50° C. for at least one hour.

8. (canceled)

9. The enveloped virus composition according to claim 1, wherein the one or more enveloped viruses comprises one or more enveloped DNA viruses, one or more enveloped RNA viruses, or a combination thereof.

10. The enveloped virus composition according to claim 1, wherein the one or more enveloped viruses comprises one or more enveloped DNA viruses comprising one or more of baculoviruses, poxviridae, fowlpox virus, herpes viruses, poxviruses, hepadnaviruses, asfarviruses, and a combination thereof.

11. The enveloped virus composition according to claim 1, wherein the one or more enveloped viruses comprises one or more enveloped RNA viruses comprising one or more of lentiviruses, flaviviruses, lyssaviruses, ephemeroviruses, vesiculovirus, alphaviruses, togaviruses, coronaviruses, hepatitis D, orthomyxoviruses, paramyxoviruses, avian paramyxoviruses rhabdovirus, bunyaviruses, influenza, hepatitis C, measles, rabies virus, Vesicular stomatitis, mumps, and Ebola virus, filoviruses and a combination thereof.

12. The enveloped virus composition according to claim 1, wherein the one or more enveloped viruses comprises one or more retroviruses; optionally, wherein the one or more retroviruses comprise one or more of a gamma-retrovirus, lentiviruses, human T-lymphotropic virus type 1 (HTLV-1), human T-lymphotropic virus type 2 (HTLV-II), or other oncoretrovirus.

13. The enveloped virus composition according to claim 1, wherein the one or more enveloped viruses comprise one or more of influenza A virus, influenza B virus, influenza C virus, influenza D virus, the respiratory syncytial virus (RSV), corona virus, hepatitis B, hepatitis C, Ebola virus or the like.

14. The enveloped virus composition according to claim 1, wherein the one or more enveloped viruses comprise lentivirus.

15. The enveloped virus composition according to claim 1, wherein the one or more amino acid comprises one or more of histidine, methionine, arginine, proline, glycine, isoleucine, and leucine.

16. The enveloped virus composition according to claim 1, further comprising a surfactant; optionally, wherein the surfactant comprises a hydrophilic nonionic surfactant.

17. The enveloped virus composition according to claim 1, wherein the composition further comprises one or more of a polysorbate and urea.

18. The enveloped virus composition according to claim 1, wherein a surfactant is specifically excluded from the composition; optionally, wherein the excluded surfactant comprises a hydrophilic nonionic surfactant.

19. The enveloped virus composition according to claim 1, wherein the one or more non-reducing saccharide agents comprises trehalose or sucrose; the one or more polysaccharides comprises HES, and the one or more amino acids comprises proline or leucine.

20. A pharmaceutical composition comprising a composition according to claim 2 of use in as a prophylactic or in a therapeutic treatment wherein the enveloped virus composition is essentially dry, and further comprises at least one pharmaceutically acceptable excipient.

21-32. (canceled)

33. A method for eliciting an immune response to one or more pathogenic organisms in a subject, the method comprising administering to the subject a reconstituted pharmaceutical composition according to claim 20 and eliciting an immune response in the subject.

34-35. (canceled)

36. A kit comprising an enveloped virus composition according to claim 1; and at least one container.
